SEQUENCE LISTING (41 sequences)

SEQ ID NO: 1 - DNA, 20 bp, synthetic construct
  source 1..20: mol_type = other DNA
cctcgagctccctctgagcc

SEQ ID NO: 2 - DNA, 1449 bp, synthetic construct
  source 1..1449: mol_type = other DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcggacccagctttcttgtacaaagtggttgatatccagcacagtggcggccgctcgagtctagagggcccgcggttcgaaggtaagcctatccctaaccctctcctcggtctcgattctacgcgtaccggttag

SEQ ID NO: 3 - protein, 393 aa, Homo sapiens [sequence omitted]

SEQ ID NO: 4 - protein, 390 aa, Mus musculus
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 5 - DNA, 30 bp, synthetic construct
  source 1..30: mol_type = other DNA
cagtgggtcagaagtttacatacactaagg

SEQ ID NO: 6 - DNA, 31 bp, synthetic construct
  source 1..31: mol_type = other DNA
cagtgggtcagaagttaacatacactcaatt

SEQ ID NO: 7 - DNA, 30 bp, synthetic construct
  source 1..30: mol_type = other DNA
agttgaatcggaagtttacatacaccttag

SEQ ID NO: 8 - DNA, 226 bp, synthetic construct
  source 1..226: mol_type = other DNA
agttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 9 - DNA, 228 bp, synthetic construct
  source 1..228: mol_type = other DNA
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaatgtatttggctaaggtgtatgtaaacttccgacttcaactg

SEQ ID NO: 10 - protein, 340 aa, synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWCGFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 11 - protein, 230 aa, synthetic construct
GPSGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 12 - protein, 230 aa, synthetic construct; identical to SEQ ID NO: 11

SEQ ID NO: 13 - protein, 346 aa, synthetic construct
MEEPQSDPSIEPPLSQETFSDLWKLLPENNVLSPLPSQAVDDLMLSPDDLAQWLTEDPGPDEAPRMSEAAPPMAPTPAAPTPAAPAPAPSWPLSSSVPSQKTYHGSYGFRLGFLHSGTAKSVTCTYSPDLNKMFCQLAKTCPVQLWVDSTPPPGSRVRAMAIYKQSQHMTEVVRRCPHHERCSDSDGLAPPQHLIRVEGNLRVEYSDDRNTFRHSVVVPYEPPEVGSDCTTIHYNYMCNSSCMGGMNRRPILTIITLEDSSGNLLGRNSFEVRVCACPGRDRRTEEENFRKKGEPCHQLPPGSTKRALPNNTSSSPQPKKKPLDGEYFTLQVLKSWDLLSSGKFLV

SEQ ID NO: 14 - DNA, 23 bp, synthetic construct
  source 1..23: mol_type = other DNA
ccggttcatgccgcccatgcagg

SEQ ID NO: 15 - DNA, 23 bp, synthetic construct
  source 1..23: mol_type = other DNA
ggcaaggctcccctttcttgcgg

SEQ ID NO: 16 - DNA, 23 bp, synthetic construct
  source 1..23: mol_type = other DNA
tgtaacagttcctgcatgggcgg

SEQ ID NO: 17 - DNA, 23 bp, synthetic construct
  source 1..23: mol_type = other DNA
tctgtgcgccggtctctcccagg

SEQ ID NO: 18 - DNA, 23 bp, synthetic construct
  source 1..23: mol_type = other DNA
gtgcgagtttgtgcctgtcctgg

SEQ ID NO: 19 - DNA, 23 bp, synthetic construct
  source 1..23: mol_type = other DNA
agagaatttccgcaagaaagtgg

SEQ ID NO: 20 - DNA, 23 bp, synthetic construct
  source 1..23: mol_type = other DNA
aattctcttcctctgtgcgccgg

SEQ ID NO: 21 - DNA, 20 bp, synthetic construct
  source 1..20: mol_type = other DNA
tcgacgctaggatctgactg

SEQ ID NO: 22 - DNA, 20 bp, synthetic construct
  source 1..20: mol_type = other DNA
tcccacctccagccctaagg

SEQ ID NO: 23 - DNA, 19 bp, synthetic construct
  source 1..19: mol_type = other DNA
ggcagctacggtttccgtc

SEQ ID NO: 24 - DNA, 20 bp, synthetic construct
  source 1..20: mol_type = other DNA
ctgtgagtggatccattgga

SEQ ID NO: 25 - DNA, 165 bp, synthetic construct
  source 1..165: mol_type = other DNA
ttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaat

SEQ ID NO: 26 - DNA, 32 bp, synthetic construct
  source 1..32: mol_type = other DNA
cagttgaagtcggaagtttacatacacttaag

SEQ ID NO: 27 - DNA, 32 bp, synthetic construct
  source 1..32: mol_type = other DNA
tccagtgggtcagaagtttacatacactaagt

SEQ ID NO: 28 - DNA, 1023 bp, synthetic construct
  source 1..1023: mol_type = other DNA
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 29 - DNA, 1023 bp, synthetic construct
  source 1..1023: mol_type = other DNA
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggtcttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 30 - protein, 340 aa, synthetic construct
MGKSKEISQDLRAKIVDLHKSGSSLGAISKRLAVPRSSVQTIVRKYKHHGTTQPSYRSGRRRVLSPRDERTLVRKVQINPRTAAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 31 - DNA, 1023 bp, synthetic construct
  source 1..1023: mol_type = other DNA
atgggaaaatcaaaagaaatcagccaagacctcagagcgaaaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacagcggcaaaggaccttgtgaagatgctggaggaaacaggcacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggtcataatgaccatcgttatgtttggaggaagaagggggaggcttgcaagccgaagaacaccatcccaaccgtgaagcacgggggtggcagcatcatgttgtgggggtgctttgccgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactag

SEQ ID NO: 32 - DNA, 29 bp, synthetic construct
  source 1..29: mol_type = other DNA
gttcaagtcggaagtttacatacacttag

SEQ ID NO: 33 - DNA, 20 bp, synthetic construct
  source 1..20: mol_type = other DNA
gctaacgatctctttgatga

SEQ ID NO: 34 - DNA, 20 bp, synthetic construct
  source 1..20: mol_type = other DNA
gaacatcgaggccaaatccc

SEQ ID NO: 35 - DNA, 20 bp, synthetic construct
  source 1..20: mol_type = other DNA
gttcggttaccgtcctgcga

SEQ ID NO: 36 - DNA, 20 bp, synthetic construct
  source 1..20: mol_type = other DNA
tcagttgttaaagcaagttg

SEQ ID NO: 37 - DNA, 20 bp, synthetic construct
  source 1..20: mol_type = other DNA
cagaaaatgacggcagcact

SEQ ID NO: 38 - DNA, 20 bp, synthetic construct
  source 1..20: mol_type = other DNA
actgcatccatatcgatgac

SEQ ID NO: 39 - DNA, 20 bp, synthetic construct
  source 1..20: mol_type = other DNA
gccgcgtttgaaccgcgatc

SEQ ID NO: 40 - DNA, 21 bp, synthetic construct
  source 1..21: mol_type = other DNA
gcgaatgatcacagcaatgaa

SEQ ID NO: 41 - DNA, 21 bp, synthetic construct
  source 1..21: mol_type = other DNA
gtggcgaatgatcacagcaat

(19) United States
(12) Patent Application Publication
(10) Pub. No.: US 2025/0064033 A1
(43) Pub. Date: Feb. 27, 2025

(54) ANIMAL MODELS OF RECCURRENT HEPATOCELLULAR CARCINOMA AND USES THEREOF

(71) Applicants: The University of Hong Kong, Hong Kong (CN); Centre for Oncology and Immunology Limited, Hong Kong (CN)

(72) Inventors: Carmen Chak Lui Wong, Hong Kong (CN); Chun Ming Wong, Hong Kong (CN); Wai Ching Chin, Hong Kong (CN); Wai Hin Yuen, Hong Kong (CN)

(21) Appl. No.: 18/815,713

(22) Filed: Aug. 26, 2024

Related U.S. Application Data
(60) Provisional application No. 63/578,576, filed on Aug. 24, 2023.

(51) Int. Cl. A01K 67/0278; C12N 9/22; C12N 15/11; C12N 15/85; G01N 33/50

(52) U.S. Cl. CPC A01K 67/0278; C12N 9/22; C12N 15/111; C12N 15/8509; G01N 33/5017; A01K 2217/206; A01K 2267/0331; C12N 2015/8572; C12N 2310/20; G01N 2500/10

(57) ABSTRACT

Compositions including recurrent hepatocellular carcinoma (HCC) model generating systems are disclosed. The HCC model generating system contains a CRISPR-Cas expression vector, a transposase expression vector, and a transposon expression vector. Also provided is a non-human animal model of recurrent HCC containing genetic modifications introduced by integration of the model generating system into target liver cells of target animals. Upon stable integration, the model generating system modifies expression of Tp53 and expresses the oncogene in the target liver cells of the target animal, resulting in the development of a HCC tumors in the liver. Methods of using the non-human animal model of recurrent HCC are also provided. The non-human animal model of recurrent HCC can be used for research purposes such as investigating the molecular and genetic mechanisms underlying recurrent HCC tumor development, identifying potential therapeutic targets, and evaluating/screening potential compounds for the treatment of recurrent HCC.

### CROSS-REFERENCE TO RELATED APPLICATIONS

This application claims benefit of and priority to U.S. Provisional Application No. 63/578,576, filed Aug. 24, 2023, the content of which is hereby incorporated herein by reference in its entirety.

### REFERENCE TO SEQUENCE LISTING

The Sequence Listing XML submitted as a file named “UHK_01255_US_ST26.xml” created on Aug. 19, 2024, and having a size of 44,357 bytes is hereby incorporated by reference pursuant to 37 C.F.R. § 1.834(c)(1).

### FIELD OF THE INVENTION

The disclosed invention is generally in the field of animal models for liver cancer and specifically in the area of animal models for hepatocellular carcinoma.

### BACKGROUND OF THE INVENTION

Hepatocellular carcinoma (HCC) is a primary malignancy of the liver which develops from transformed hepatocytes. HCC accounts for 90% of primary liver cancers, representing the fifth most common, and second most lethal cancer worldwide. The high mortality rate associated with HCC is attributed to its high recurrence and increased resistance to chemo-, radio-, and targeted-therapy. Treatments for recurrent HCC are very limited and ineffective. To date, the treatment options available include surgical treatments such as ablation, resection, and liver transplantation. Surgical resection is currently the most effective treatment for HCC. However, more than 70% of patients experience recurrence of HCC tumors within 5 years following surgery, representing the major cause of mortality in HCC patients. Currently, there is no promising curative therapy for HCC patients suffering from recurrence of HCC tumors. As a result, patients with recurrent HCC are deemed incurable, generally succumbing to the disease in a short period. Unfortunately, the prognosis of recurrent HCC is lower than that for primary HCC.

Despite the unmet clinical need for recurrent HCC, most basic and translational research studies currently focus on primary HCC but not recurrent HCC. To date, existing clinical trials only focus on the efficiency of tyrosine kinase inhibitors (TKI, sorafenib) and immune checkpoint inhibitors (ICI, anti-PD-1/PD-L1) as adjuvant therapies for recurrent HCC in patients who underwent surgical treatments. However, new therapeutic regimens to effectively control recurrent HCCs still await validation.

Understanding of the molecular mechanisms driving HCC recurrence is scarce due to a combination of factors. First, there is no cell line that truly distinguishes recurrent HCC from primary HCC. Second, there is no reliable translational mouse model with an intact immune system that could recapitulate the process of HCC recurrence. Third, liver surgery in small animals is complicated and technically challenging. Orthotopic implantation of human HCC cells in immune-deficient mice could generate focal HCC tumors that are clinically similar to resectable HCC. However, this can only be achieved in immune-compromised animals. Syngeneic mouse HCC cell lines, such as Hepa1-6, after orthotopic implantation in immune competent mice regress and fail to develop recurrent tumors. Further, Hepatocarcinogen (DEN)-induced mouse HCC or hydrodynamic tail vein injection (HDTVi)-mediated mouse HCC were established systemically, but the precise tumor location and numbers formed in the liver cannot be controlled. Resection could only be performed in patients with good liver function with preferably smaller tumor size and fewer nodules. Similar to HCC patients, surgeries become dangerous when multiple lobes have to be removed in the mice as the liver is a critical organ. Thus, there is an urgent need for more clinically translatable models of recurrent HCC for biomarker and therapeutic target identification.

An object of the present invention is to provide compositions for generating animal models of recurrent hepatocellular carcinoma (HCC).

It is also an object of the present invention to provide an animal model of recurrent HCC for the study of HCC biology, evaluation of new and existing treatments, and development of personalized treatment strategies.

Another object of the present invention is to provide methods of generating a animal models of recurrent HCC.

It is also an object of the present invention to provide methods of using recurrent HCC animal models for biomarker identification and treatment evaluation.

### BRIEF SUMMARY OF THE INVENTION

Compositions including recurrent hepatocellular carcinoma (HCC) model generating systems and non-human animal models of recurrent HCC are disclosed. The compositions are suitable for investigation and identification of biomarkers that predict tumor recurrence, identification of molecular targets for the treatment of recurrent HCC, evaluation of therapies for recurrent HCC, and the investigation of the mechanisms underlying HCC recurrence.

The compositions generally include a recurrent hepatocellular carcinoma (HCC) model generating system containing a CRISPR-Cas expression vector, a transposon expression vector, and a transposase expression vector. The CRISPR-Cas expression vector generally includes a nucleic acid sequence encoding a CRISPR-associated (Cas) endonuclease, a nucleic acid sequence encoding a single guide RNA (sgRNA), and a delivery vector carrier that facilitates the delivery and expression of the sequences encoding the Cas endonuclease and the sgRNA in target liver cells. In some forms, the sgRNA is designed to specifically target and bind to a predetermined sequence within the Tumor protein p53 (TP53) oncogene. In some forms, the sgRNA which targets and binds to a predetermined sequence in the first oncogene has the sequence:

(SEQ ID NO: 1)
CCTCGAGCTCCCTCTGAGCC.

In some forms, Cas endonuclease is selected from the group containing Cas9, Cas12a, Cas12b, or Cas13.

In some forms, the CRISPR-Cas expression vector further contains one or more transcriptional mediation sequences selected from the group containing nuclear localization sequences, promoter sequences, enhancer sequences, marker sequences, termination signal sequences, polyadenylation signal sequences, and splicing signal sequences. In some forms, the delivery vector carrier is a plasmid, viral vector, or non-viral vector. In some forms, the CRISPR-Cas expression vector further contains one or more selectable markers for identifying and isolating transfected or transduced cells.

The model generating system also contains a transposase expression vector which generally includes a nucleic acid sequence encoding a transposase; and a first antibiotic resistance gene for the selection of cells containing the transposase expression vector. The transposase expression vector is designed to lead to expression of the transposase in the target liver cells. In some forms, the transposase is selected from the group consisting of Sleeping Beauty, piggyBac, Tn5, Tn7, and Mu.

The model generating system also contains a transposon expression vector which generally includes a nucleic acid sequence encoding an oncogene; a pair of inverted terminal repeat (ITR) sequences located on either side of the sequence encoding the oncogene, wherein the ITR sequences are recognized and bound by the transposase; and a second antibiotic resistance gene for the selection of cells containing the transposon expression vector. Delivery of the transposon expression vector is designed to lead to expression of the oncogene in the target liver cells. In some forms, the inverted terminal repeat sequences are selected from the group containing Sleeping Beauty, piggyBac, Tn5, Tn7, and Mu ITR sequences. In some forms, the oncogene is cellular myelocytomatosis (c-myc). In some forms, the c-Myc oncogene contains SEQ ID NO:2 or a variant thereof. In some forms, delivery of the transposon expression vector to the host cell increases c-myc expression in the host cell.

In some forms, the first and second antibiotic resistance genes are independently selected from the group containing ampicillin, kanamycin, tetracycline, and chloramphenicol resistance genes.

In some forms, the transposon expression vector further contains a regulatory element for controlling the expression of the oncogene in a host cell, wherein the regulatory element is selected from the group consisting of promoters, enhancers, silencers, and insulators. In some forms, the promoter is a liver-specific promoter selected from the group containing albumin promoter, the alpha-1 antitrypsin promoter, the apolipoprotein E promoter, alpha-fetoprotein (AFP) promoter, CYP1A1 promoter, CYP2B6 promoter, CYP3A4 promoter, fatty acid-binding protein (FABP) promoter, glucose-6-phosphatase (G6Pase) promoter, Hepatitis B virus (HBV) core promoter, HNF1-alpha promoter, HNF4-alpha promoter, Interleukin-6 (IL-6) promoter, and Transferrin promoter.

In some forms, the transposon expression vector further contains a reporter gene sequence located within the ITR sequence for monitoring the expression of the oncogene in a host cell. In some forms, the reporter gene sequence encodes a protein selected from the group consisting of green fluorescent protein (GFP), red fluorescent protein (RFP), luciferase, and β-galactosidase.

Also provided is a non-human animal model of recurrent hepatocellular carcinoma (HCC). The non-human animal model generally contains a genetic modification introduced by integration of the model generating system into the target liver cells of the target animal. When the model generating system is stably integrated into the target liver cells of the target animal, the model generating system modifies expression of Tp53 and expresses the oncogene in the target liver cells of the target animal, resulting in the development of a focal HCC tumor in the liver of the target animal.

In some forms, the period of time for the development of a focal HCC tumor is about 4 weeks. In some forms, the non-human animal model develops one or more recurrent HCC liver tumors after a period of time following surgical resection of the focal tumors. In some forms, the period of time for development of the recurrent HCC tumors is from about 6 weeks to about 6 months following surgical resection of the focal tumors.

In some forms, the target animal is a mammal, preferably mice, rats, or other rodents. In preferred forms, the target animal is a mouse or rat, more preferably mice. In these forms, the mice are preferably immune-competent mice.

Methods of generating a non-human animal model of recurrent hepatocellular carcinoma (HCC) is also provided. An exemplary method includes one or more of the following steps:
(i) injecting pre-determined amounts of the components of the model generating system into hepatocytes of the target animal;
(ii) applying an electric pulse via electroporation to the site of injection to facilitate transfection of the model generating system into the hepatocytes, wherein conditions of the electroporation are optimized to maximize transfection efficiency;
(iii) allowing the target animal to recover for a period of time during which a focal HCC tumor is generated;
(iv) resecting the focal HCC tumor; and
(v) allowing the target animal to recover for a period of time during which recurrent HCC tumors are established.

In some forms, the components of the model generating system injected into the target animal in a ratio of about 10:5:1 for the CRISPR-Cas expression vector, the transposon expression vector, and the transposase expression vector, respectively.

In some forms, the model generating system is delivered to the hepatocytes via electroporation. In some forms, the electroporation is performed in two steps: a poring pulse mode and a transfer pulse mode. In some forms, the poring pulse mode of electroporation is performed at a voltage of between about 1 and about 100 volts, a pulse duration of between about 1 and about 50 milliseconds, a pulse interval of between about 1 and about 50 m/s, a pulse number of between about 1 and about 5, and a decay rate of between about 5 and about 15%. In some forms, the transfer pulse mode of electroporation is performed at a voltage of between about 1 and about 100 volts, a pulse duration of between about 1 and about 50 milliseconds, a pulse interval of between about 1 and about 50 m/s, a pulse number of between about 3 and about 8, and a decay rate of between about 30 and about 50%.

Methods of using the non-human animal model of recurrent HCC are also provided. The non-human animal model of recurrent HCC can be used for research purposes such as investigating the molecular and genetic mechanisms underlying recurrent HCC tumor development, identifying potential therapeutic targets, and evaluating/screening potential compounds for the treatment of recurrent HCC. The disclosed non-human animal model is also valuable for the collection of liver cells and/or tissues containing recurrent HCC tumors for research purposes.

An exemplary method of screening and evaluating compounds for treating recurrent hepatocellular carcinoma (HCC), includes one or more of the following steps:
(i) administering a pre-determined concentration of a test compound to the non-human animal or to HCC tumor cells harvested from the non-human animal;
(ii) incubating the animal or cells for a pre-determined period of time;
(iii) measuring one or more parameters indicative of cellular response to the test compound; and
(iv) assessing the efficacy of the test compound by comparing the measured parameters of step (iii) to measured parameters of an untreated control non-human animal, wherein a significant difference between the measured parameters of step (iii) and the measured parameters of the untreated control indicates that the test compound can be considered for treating recurrent HCC.

In some forms, the one or more parameters indicative of cellular response are selected from the group containing cell viability, cell proliferation, cell death, metabolic activity, gene expression, protein expression, and any combination thereof. In some forms, the measuring of the one or more parameters in step (iii) is performed using one or more techniques selected from the group of flow cytometry, microscopy, gene expression analysis, quantitative polymerase chain reaction (qPCR), enzyme-linked immunosorbent assay (ELISA), mass spectrometry, and any combination thereof.

In some forms, the predetermined concentration and predetermined period of time in steps (i) and (ii), respectively, are optimized based on the type of test compound.

In some forms, the test compound is selected from a library of compounds containing natural products, synthetic compounds, or a combination thereof.

An exemplary method for identifying biomarkers of hepatocellular carcinoma (HCC), the method includes one or more of the following steps:
(i) extracting recurrent HCC tumor tissue from the non-human animal model;
(ii) isolating and purifying nucleic acids, proteins, and/or metabolites from the recurrent HCC tumor tissue;
(iii) measuring the expression level of the nucleic acids, proteins, and/or metabolites;
(iv) comparing the expression levels of the nucleic acids, proteins, and/or metabolites to corresponding expression levels in the nucleic acids, proteins, and/or metabolites of control liver issue; and
(v) identifying one or more biomarkers of HCC based on statistically significant differences in expression levels of the nucleic acids, proteins, and/or metabolites between the recurrent HCC tumor tissue and the control liver issue.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 is a schematic of the workflow for the experiments.

FIGS. 2A-2C show data of an example of the disclosed HCC mouse model. FIG. 2A is a collection of photos showing the recurrent HCC mouse model: Genome-editing plasmids were electroporated specifically into the left lobe of the liver. Focal tumors were formed. Hepatectomy was performed to surgically remove focal HCC (Tp53^{KO}cMyc^{OE}). Recurrent tumor was formed within 1-month post-resection. FIG. 2B is a line graph showing disease-free survival of mice post-resection. Surgical resection was performed in mice carrying Tp53^{KO}cMyc^{OE} and non-recurrent genotype tumors of similar size. FIG. 2C is a line graph of TCGA data showing that HCC patients with TP53 mutations have poorer disease-free survival post-resection as compared to HCC patients with TP53 WT. >400 HCC patients were included in the analysis.

FIGS. 3A and 3B show that cold tumors (Few CD8⁺ T cell infiltration) tend to recur rapidly while hot tumors (High CD8+ T cell infiltration) have a lower recurrent rate after surgical resection. The livers of wild-type C57BL/6 mice were injected with genome-editing plasmid mix. The plasmid contains sgRNA targeting Trp53/Keap1/Pten or contains CTNNB1 gene. The injected site was electroporated to initiate tumorigenesis. Primary tumors were resected to make formalin-fixed paraffin-embedded (FFPE) tissues. Immunohistochemistry of these FFPE tissue sections using anti-CD8a antibody has revealed the number of CD8+ T cell infiltration in primary tumors. After resection of primary tumors, the recurrence-free survival was monitored. FIG. 3A are images of CD8 staining (left panel) performed in Trp53^{KO}/MYC^{OE} and Keap1^{KO}/MYC^{OE} liver cancer (HCC) resected primary tumor. CD8+ T cells were counted and results are shown (right panel). FIG. 3B is a survival analysis of mice with Trp53^{KO}/MYC^{OE} (cold), Pten^{KO}/MYC^{OE} (cold), CTNNB1^{OE}/MYC^{OE} (cold), Keap1^{OE}/MYC^{OE} (hot) primary liver cancer after tumor resection.

### DETAILED DESCRIPTION OF THE INVENTION

The disclosed method and compositions can be understood more readily by reference to the following detailed description of particular embodiments and the Example included therein and to the Figures and their previous and following description.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. DEFINITIONS

As used herein, the term “hepatocellular tumor” refers to a group of cells which are committed to a hepatocellular lineage, and which exhibit an altered growth phenotype. The term encompasses tumors that are associated with hepatocellular malignancy (i.e., HCC) as well as with pre-malignant conditions such as hepatoproliferative and hepatocellular hyperplasia and hepatocellular adenoma, which include proliferative lesions that are perceived to be secondary responses to degenerative changes in the liver.

As used herein, an “altered hepatocyte” refers to a change in the level of a gene and/or gene product with respect to any one of its measurable activities in a hepatocyte (e.g., the function which it performs and the way in which it does so, including chemical or structural differences and/or differences in binding or association with other factors). An altered hepatocyte may be affected by one or more structural changes to the nucleic acid or polypeptide sequence, a chemical modification, an altered association with itself or another cellular component or an altered subcellular localization. For example, an altered hepatocyte may have “activated” or “increased” expression of an oncogene, “repressed” or “decreased” expression of a tumor suppressor gene or both.

As used herein, the term “gene” refers to a nucleic acid (e.g., DNA or RNA) sequence that including coding sequences necessary for the production of a polypeptide, RNA (e.g., including, but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation.

As used herein, “mammal” includes both humans and non-humans and include but is not limited to humans, non-human primates, canines, felines, murines, bovines, equines, and porcines.

The terms “target site” or “target sequence,” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “noncomplementary strand” or “non-complementary strand.” By “site-directed modifying polypeptide” or “RNA-binding site-directed polypeptide” or “RNA-binding site-directed modifying polypeptide” or “site-directed polypeptide” is meant a polypeptide that binds RNA and is targeted to a specific DNA sequence. A site-directed modifying polypeptide as described herein is targeted to a specific DNA sequence by the RNA molecule to which it is bound. The RNA molecule contains a sequence that binds, hybridizes to, or is complementary to a target sequence within the target DNA, thus targeting the bound polypeptide to a specific location within the target DNA (the target sequence).

“Nuclease” and “endonuclease” are used interchangeably herein to mean an enzyme which possesses endonucleolytic catalytic activity for DNA cleavage.

The term “transposon,” as used herein, refers to a nucleic acid segment, which is recognized by a transposase or an integrase enzyme and which is an essential component of a functional nucleic acid-protein complex (i.e., a transpososome) capable of transposition. The term “transposase binding sequence,” “transposase binding site,” “transposon joining strand” or “joining end,” as used herein refers to the nucleotide sequences that is always within the transposon end sequence to which a transposase specifically binds when mediating transposition. The transposase binding sequence may however contain more than one site for the binding of transposase subunits. The expression “transposition reaction” used herein refers to a reaction wherein a transposon inserts into a target nucleic acid. Primary components in a transposition reaction are a transposon and a transposase or an integrase enzyme.

As used herein, a “vector” is a replicon, such as a plasmid, phage, or cosmid, into which another DNA segment may be inserted so as to bring about the replication of the inserted segment. The vectors described herein can be expression vectors. A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that contain a population of daughter cells containing the transforming DNA. A “clone” is a population of cells derived from a single cell or common ancestor by mitosis. A “cell line” is a clone of a primary cell that is capable of stable growth in vitro for many generations.

As used herein, an “expression vector” is a vector that includes one or more expression control sequences.

As used herein, an “expression control sequence” is a DNA sequence that controls and regulates the transcription and/or translation of another DNA sequence.

As used herein, the term “treating” includes alleviating the symptoms associated with a specific disorder or condition and/or preventing or eliminating the symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, “transformed” and “transfected” encompass the introduction of a nucleic acid (e.g., a vector) into a cell by a number of techniques known in the art.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression, or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with, or restraining the expression and/or activity of the gene/gene product pathway relative to a standard or a control.

The term “fragment” means a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

The terms “isolated,” “purified,” or “biologically pure” mean material that is free to varying degrees from components which normally accompany it as found in its native state. “Isolate” denotes a degree of separation from original source or surroundings. “Purify” denotes a degree of separation that is higher than isolation.

The term “isolated nucleic acid” refers to a nucleic acid that is separated from other nucleic acid molecules that are present in a mammalian genome, including nucleic acids that normally flank one or both sides of the nucleic acid in a mammalian genome. An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

“Variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. The term “polypeptides” includes proteins and fragments thereof. By “protein” or “polypeptide” or “peptide” is meant any chain of more than two natural or unnatural amino acids, regardless of post-translational modification (e.g., glycosylation or phosphorylation), constituting all or part of a naturally occurring or non-naturally occurring polypeptide or peptide, as is described herein. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It can be appreciated that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid (e.g., a plasmid or recombinant expression vector) and a eukaryotic host cell is a genetically modified eukaryotic host cell (e.g., a mammalian somatic cell), by virtue of introduction into a suitable eukaryotic host cell of an exogenous nucleic acid.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. COMPOSITIONS

Compositions including recurrent hepatocellular carcinoma (HCC) model generating systems and non-human animal models of recurrent HCC have been established. The compositions are suitable for investigation and identification of biomarkers that predict tumor recurrence, identification of molecular targets for the treatment of recurrent HCC, evaluation of therapies for recurrent HCC, and the investigation of the mechanisms underlying HCC recurrence.

A. Model-Generating System for Hepatocellular Carcinoma (HCC) As described in the non-limiting Examples, electroporation of genome-editing plasmids in the liver to inhibit expression of the tumor suppressor gene Tp53, and increase expression of the oncogene, cMyc, allowed the generation of focal HCC tumors in specific regions of the liver. Further, it was established that recurrent HCC tumors consistently developed in Tp53^{KO}cMyc^{OE} tumor tissues within one month following surgical resection of focal HCC tumors.

Thus, a model generating system for recurrent HCC is disclosed. The model generating system alters target liver cells to increase oncogene expression and reduce tumor suppressor gene expression. The model generating system leverages multiple gene engineering techniques to make precise modifications to the DNA of non-reproductive (somatic) cells within an organism. This contrasts with germline editing, which involves making changes to the DNA of reproductive cells, which would be passed on to filial generations. The disclosed model generating system generally contains a CRISPR-Cas expression vector, a transposase expression vector, and a transposon expression vector.

### 1. CRISPR-Cas Expression Vector

The model generating system typically contains a CRISPR-Cas expression vector. The CRISPR-Cas system is a defense mechanism found in bacteria and archaea that enables them to recognize, target, and destroy foreign genetic material, such as viruses and plasmids. Generally, the CRISPR-Cas expression vector contains a CRISPR-associated (Cas) endonuclease enzyme, a single guide RNA (sgRNA) molecule, and a delivery vector carrier. Typically, the combination of the Cas endonuclease and the sgRNA molecule leads to inhibition of the expression of tumor suppressor genes in target liver cells.

The CRISPR-Cas expression vector includes one or more Cas (CRISPR associated) proteins to mediate processing of CRISPR loci transcripts, cleavage of the target DNA or RNA, and new spacer integration. In some forms, the Cas protein is Cas5, Cas6, Cas7, Cas8, Cas9, Cas12a, Cas12b, and Cas13, or any combination thereof. In some forms, the Cas protein is a recombinant Cas protein having from about 75% to about 100%, from about 80% to about 100%, from about 90% to about 100%, or from about 95% to about 100% sequence identity to a Cas5, Cas6, Cas7, Cas8, Cas9, Cas12a, Cas12b, Cas13 protein, or any combination thereof.

In preferred forms, the Cas protein is Cas9. The structure and mechanism of function of Cas9 proteins are known in the art. Briefly, the Cas9 protein is a bi-lobed structure, having an alpha helical recognition (REC) lobe and a nuclease (NUC) lobe. The REC lobe recognizes the target DNA by finding the complementary DNA region, and the NUC lobe cleaves the double stranded DNA by RNA-guided dsDNA endonuclease activity (Jiang and Doudna, Annual Review of Biophysics, 46:505-529 (2017)). Cas9 proteins generally have six domains: a REC I domain, a REC II domain, a Bridge Helix domain, a PAM Interacting domain, a HNH domain and a RuvC domain (Jinek, et al., Science, 343(6176):1247997 (2014) and Nishimasu et al., Cell, 156(5):935-49 (2014)). The REC I domain, the largest domain, spans residues 94-179 and 308-713, and facilitates RNA guided DNA targeting. The arginine-rich bridge helix spans residues 60-93 and initiates cleavage activity upon binding of target DNA (Nishimasu et al., Cell, 156(5):935-49 (2014)). The PAM-Interacting domain spans residues 1099-1368 finds the PAM sequence on the target DNA and initiates binding to the target DNA (Anders et al., Nature, 513(7519):569-73 (2014); Nishimasu et al., Cell, 156(5):935-49 (2014), Sternberg et al., Nature, 507(7490):62-7 (2014)). The HNH and RuvC domains are nuclease domains that cut single-stranded DNA (Jinek, et al., Science, 343(6176):1247997 (2014) and Nishimasu et al., Cell, 156(5):935-49 (2014)). The RuvC domain, including RuvCI, RuvCII, and RuvCIII, span residues 1-59, 718-769, and 909-1098, and have RNase H activity, and cuts single-stranded DNA for the non-complementary strand of the target DNA. The HNH domain spans residues 775-908 and cuts single-stranded DNA for the complementary strand of the target DNA. The structure of Cas9 proteins are described in further details in Jiang and Doudna, Annual Review of Biophysics, 46:505-529 (2017); Jiang and Doudna, Curr Opin Struct Biol, 30:100-111 (2015); Huai et al., Nature Communications, 8(1):1375 (2017), all of which are incorporated herein by reference in their entireties.

In some forms, the Cas9 protein is derived from Streptococcus pyogenes, Streptococcus aureus, Campylobacter jejuni, Streptococcus thermophilus, or Treponema denticola. Thus, in some forms, the Cas9 protein is a Streptococcus pyogenes Cas9 (SpCas9), Campylobacter jejuni Cas9 (CjCas9), Streptococcus canis (ScCas9), Streptococcus thermophilus Cas9 (StCas9), Staphylococcus aureus Cas9 (SaCas9), and Neisseria meningitides Cas9 (NmCas9), or any variant thereof.

In some forms, the Cas9 protein is a naturally occurring Cas9 protein. Exemplary naturally occurring Cas9 proteins include but are not limited to SpCas9, ScCas9, and SaCas9. In some forms, the Cas9 protein is a variant of a naturally occurring Cas9 protein. Examples of suitable variants of SpCas9 proteins include but are not limited to SpCas9-NRRH, SpG, SpCas9-NRCH, SpCas9-NRTH, and variants thereof. Examples of suitable variants of SaCas9 proteins include but are not limited to efSaCas9, KKHSaCas9, SaCas9-HF, and variants thereof. Examples of suitable variants of ScCas9 proteins include but are not limited to SpCas9++, SpCas9n++, SpCas9+, and variants thereof.

In some forms, the Cas9 protein is an artificially engineered Cas9 protein. Exemplary artificially engineered Cas9 proteins include but are not limited to XCas9, ThermoCas9, dCas9, eSpCas9, Cas9-DD, SpCas9-VQR, SpCas9-EQR, and HypaCas9.

In some forms, the Cas9 protein is a variant of an artificially engineered Cas9 protein. In some forms, a variant is at least about 70%, about 80%, about 85%, about 90%, about 95 identical to an artificially engineered Cas9 protein.

In some forms, the Cas9 protein requires the presence of a protospacer adjacent motif (PAM) directly downstream of the target sequence in the genomic DNA, on the non-target strand (Karvelis et al., Genome Biology, 16: Article number 253 (2015)). In some forms, when the Cas9 protein is derived from Streptococcus pyogenes, the PAM motif is NGG or NAG, preferably 5′-NGG-3′, where “N” represents any nucleotide. In some forms, when the Cas9 protein is derived from Streptococcus thermophilus, the PAM motif is NGGNG, where “N” represents any nucleotide. In some forms, when the Cas9 protein is derived from Staphylococcus aureus, the PAM motif is NNGRRT or NNGRR(N), where “N” represents any nucleotide. In some forms, when the Cas9 protein is derived from Neisseria meningitides, the PAM motif is NNNNGATT or NNNNGCTT, where “N” represents any nucleotide. In some forms, when the Cas9 protein is derived from Streptococcus canis, the PAM motif is NNG, where “N” represents any nucleotide. In some forms, when the Cas9 protein is derived from Geobacillus thermodenitrificans T12 e.g., ThermoCas9, the PAM motif is CRAA, where “R” is A or G.

The Cas9 protein in the CRISPR-Cas expression vector requires a guide RNA i.e., a single strand of RNA that forms a T-shape containing one tetraloop and two or three stem loops to become active and perform its catalytic functions. The guide RNA is engineered to have a 5′ end that is complimentary to the target DNA sequence (Jinek, et al., Science, 343(6176):1247997 (2014) and Nishimasu et al., Cell, 156(5):935-49 (2014)). This artificial guide RNA binds to the Cas9 protein and, upon binding, induces a conformational change in the protein. The conformational change converts the inactive Cas9 protein into its active form. Theoretically, steric interactions or weak binding between protein side chains and RNA bases may induce the conformational change. Once the Cas9 protein is activated, it stochastically searches for target DNA by binding with sequences that match its protospacer adjacent motif (PAM) sequence (Sternberg et al., Nature, 507(7490):62-7 (2014)). Exemplary PAMs are provided above. When the Cas9 protein finds a potential target sequence with the appropriate PAM, the protein will melt the bases immediately upstream of the PAM and pair them with the complementary region on the guide RNA (Sternberg et al., Nature, 507(7490):62-7 (2014)). If the complementary region and the target region pair properly, the RuvC and HNH nuclease domains will cut the target DNA after the third nucleotide base upstream of the PAM (Anders et al., Nature, 513(7519):569-73 (2014). The mechanism of function of Cas9 proteins are described in further details in Jiang and Doudna, Annual Review of Biophysics, 46:505-529 (2017); Jiang and Doudna, Curr Opin Struct Biol, 30:100-111 (2015); Huai et al., Nature Communications, 8(1):1375 (2017), all of which are incorporated herein by reference in their entireties.

The disclosed sgRNA molecule is designed to specifically target and bind to a predetermined sequence within a tumor suppressor gene. In some forms, the sgRNA is a synthetic RNA containing a targeting sequence and scaffold sequence derived from endogenous bacterial crRNA and tracrRNA. In some forms, the sgRNA is designed to specifically target and bind to a predetermined sequence within the TP53 oncogene. In some forms, the sgRNA which targets and binds to a predetermined sequence in the TP53 oncogene having the sequence CCTCGAGCTCCCTCTGAGCC (SEQ ID NO:1), or a variant of.

In some forms, the sgRNA is designed to specifically target and bind to a predetermined sequence within the Tp53 gene of a target non-human animal. In some forms, the combination of the Cas endonuclease and the sgRNA in the target liver cells of the target animal, leads to inhibition of Tp53 expression in the target liver cells. In some forms, the sgRNA molecule contains SEQ ID NO:1 or a variant thereof.

In some forms, the tumor suppressor gene to which the sgRNA molecule binds is the Tp53 gene. The Tp53 gene plays a critical role in regulating cell growth and preventing the development of cancer. When the Tp53 gene is mutated, it can no longer function properly, leading to uncontrolled cell growth and the development of cancer. The encoded TP53 protein responds to diverse cellular stresses to regulate expression of target genes, thereby inducing cell cycle arrest, apoptosis, senescence, DNA repair, or changes in metabolism. The canonical amino acid sequence for the human TP53 protein is represented by SEQ ID NO:3. The amino acid sequence for the mouse TP53 protein is represented by SEQ ID NO:4. The amino acid sequences for Cynomolgus monkey TP53 protein is represented by SEQ ID NO:13.

Human TP53 (Uniprot ID: P04637):
(SEQ ID NO: 3)
MEEPQSDPSVEPPLSQETFSDLWKLLPENNVLSPLPSQAMDDLMLSPDD
IEQWFTEDPGPDEAPRMPEAAPPVAPAPAAPTPAAPAPAPSWPLSSSVP
SQKTYQGSYGFRLGFLHSGTAKSVTCTYSPALNKMFCQLAKTCPVQLWV
DSTPPPGTRVRAMAIYKQSQHMTEVVRRCPHHERCSDSDGLAPPQHLIR
VEGNLRVEYLDDRNTFRHSVVVPYEPPEVGSDCTTIHYNYMCNSSCMGG
MNRRPILTIITLEDSSGNLLGRNSFEVRVCACPGRDRRTEEENLRKKGE
PHHELPPGSTKRALPNNTSSSPQPKKKPLDGEYFTLQIRGRERFEMFRE
LNEALELKDAQAGKEPGGSRAHSSHLKSKKGQSTSRHKKLMFKTEGPDS
D.
Mouse TP53 (Uniprot ID: P02340):
(SEQ ID NO: 4)
MTAMEESQSDISLELPLSQETFSGLWKLLPPEDILPSPHCMDDLLLPQD
VEEFFEGPSEALRVSGAPAAQDPVTETPGPVAPAPATPWPLSSFVPSQK
TYQGNYGFHLGFLQSGTAKSVMCTYSPPLNKLFCQLAKTCPVQLWVSAT
PPAGSRVRAMAIYKKSQHMTEVVRRCPHHERCSDGDGLAPPQHLIRVEG
NLYPEYLEDRQTFRHSVVVPYEPPEAGSEYTTIHYKYMCNSSCMGGMNR
RPILTIITLEDSSGNLLGRDSFEVRVCACPGRDRRTEEENFRKKEVLCP
ELPPGSAKRALPTCTSASPPQKKKPLDGEYFTLKIRGRKRFEMFRELNE
ALELKDAHATEESGDSRAHSSYLKTKKGQSTSRHKKTMVKKVGPDSD.
Cynomolgus monkey TP53 (Uniprot ID: A0A2K5WN49):
(SEQ ID NO: 13)
MEEPQSDPSIEPPLSQETFSDLWKLLPENNVLSPLPSQAVDDLMLSPDD
LAQWLTEDPGPDEAPRMSEAAPPMAPTPAAPTPAAPAPAPSWPLSSSVP
SQKTYHGSYGFRLGFLHSGTAKSVTCTYSPDLNKMFCQLAKTCPVQLWV
DSTPPPGSRVRAMAIYKQSQHMTEVVRRCPHHERCSDSDGLAPPQHLIR
VEGNLRVEYSDDRNTFRHSVVVPYEPPEVGSDCTTIHYNYMCNSSCMGG
MNRRPILTIITLEDSSGNLLGRNSFEVRVCACPGRDRRTEEENFRKKGE
PCHQLPPGSTKRALPNNTSSSPQPKKKPLDGEYFTLQVLKSWDLLSSGK
FLV.

In some forms, the sgRNA sequence targeting the Tp53 gene is about 17 to about 24 nucleotides in length. In some forms, the sgRNA is about 18 nucleotides, about 19 nucleotides, about 20 nucleotides, about 21 nucleotides, about 22 nucleotides, about 23 nucleotides, or about 24 nucleotides in length. In some forms, the Tp53 sgRNA sequence is CCTCGAGCTCCCTCTGAGCC (SEQ ID NO:1) or a variant thereof. In some forms, the Tp53 sgRNA sequence is:

(SEQ ID NO: 14)
CCGGTTCATGCCGCCCATGC(AGG),
(SEQ ID NO: 15)
GGCAAGGCTCCCCTTTCTTG(CGG);
(SEQ ID NO: 16)
TGTAACAGTTCCTGCATGGG(CGG);
(SEQ ID NO: 17)
TCTGTGCGCCGGTCTCTCCC(AGG);
(SEQ ID NO: 18)
GTGCGAGTTTGTGCCTGTCC(TGG);
(SEQ ID NO: 19)
AGAGAATTTCCGCAAGAAAG(TGG);
or
(SEQ ID NO: 20)
AATTCTCTTCCTCTGTGCGC(CGG),

wherein the bracket is provided with the canonical PAM (protospacer-adjacent motif) NGG at the 3′ end of the sequence, wherein “N” represents any nucleotide”. In some forms, the Tp53 sgRNA sequence is TCGACGCTAGGATCTGACTG (SEQ ID NO:21), TCCCACCTCCAGCCCTAAGG (SEQ ID NO:22), GGCAGCTACGGTTTCCGTC (SEQ ID NO:23), or CTGTGAGTGGATCCATTGGA (SEQ ID NO:24).

Variant sgRNA sequences include sequences that differ by one or more nucleotide substitutions, additions, or deletions, such as allelic variants. In some forms, the sgRNA is at least 70%, 80%, 85%, 90%, 95 identical to SEQ ID NO:1. An sgRNA sequence that is 97%, 98%, 99%, or 100% identical is also contemplated. In some forms, the Tp53 sgRNA sequence is derived from SEQ ID NO:3 or SEQ ID NO:4 or a variant thereof.

In some forms, the tumor suppressor gene to which the sgRNA molecule binds is a tumor suppressor gene associated with hepatocellular carcinoma. Exemplary tumor suppressor genes to which the sgRNA molecule binds include P53 (Tumor protein p53), PTEN (phosphatase and tensin homolog), AXIN1 (axin1), APC (APC Regulator of WNT Signaling Pathway), KEAP1 (Kelch Like ECH Associated Protein), TSC1 (TSC Complex Subunit 1), TSC2 (TSC Subunit 2), ARID1A (AT-Rich Interaction Domain 1A).

In some forms, the sgRNA is designed to specifically target and bind to a predetermined sequence within the Pten gene of a target non-human animal. In some forms, the combination of the Cas endonuclease and the sgRNA in the target liver cells of the target animal, leads to inhibition of Pten expression in the target liver cells. In some forms, the sgRNA molecule contains the sequence: GCTAACGATCTCTTTGATGA (SEQ ID NO:33) or a variant thereof.

In some forms, the sgRNA is designed to specifically target and bind to a predetermined sequence within the Tsc2 gene of a target non-human animal. In some forms, the combination of the Cas endonuclease and the sgRNA in the target liver cells of the target animal, leads to inhibition of Tsc2 expression in the target liver cells. In some forms, the sgRNA molecule contains the sequence GAACATCGAGGCCAAATCCC (SEQ ID NO:34) or a variant thereof.

In some forms, the sgRNA is designed to specifically target and bind to a predetermined sequence within the Keap1 gene of a target non-human animal. In some forms, the combination of the Cas endonuclease and the sgRNA in the target liver cells of the target animal, leads to inhibition of Keap1 expression in the target liver cells. In some forms, the sgRNA molecule contains any one of the sequences: GTTCGGTTACCGTCCTGCGA (SEQ ID NO:35), GCGAATGATCACAGCAATGAA (SEQ ID NO:40), GTGGCGAATGATCACAGCAAT (SEQ ID NO:41) or a variant thereof.

In some forms, the sgRNA is designed to specifically target and bind to a predetermined sequence within the Apc gene of a target non-human animal. In some forms, the combination of the Cas endonuclease and the sgRNA in the target liver cells of the target animal, leads to inhibition of Apc expression in the target liver cells. In some forms, the sgRNA molecule contains the sequence TCAGTTGTTAAAGCAAGTTG (SEQ ID NO:36) or a variant thereof.

The CRISPR-Cas expression vector also includes a delivery vector carrier. In some forms, the delivery vector carrier is a plasmid vector. A plasmid vector is one where a plasmid, which was found as an extranuclear circular gene that is replicated and retained outside the E. coli chromosome, is used as a vector. A plasmid vector, even with insertion of a gene of interest, may easily be multiplied within E. coli and thus has commonly been used as a gene introduction vector for animal cells. However, a plasmid vector, which is a DNA per se, is difficult to be introduced into cells without physical treatment such as microinjection or electroporation. Therefore, in some forms, a physical delivery system is used to deliver the CRISPR-Cas and sgRNA containing vector into the cells of interest. Physical delivery methods that can be used to facilitate transfer of the CRISPR-Cas expression vector include but are not limited to needles, particle bombardment, microprojectile gene transfer or gene guns, electroporation, sonoporation, photoporation, magnetofection, and hydroporation, and mechanical massage. Preferably, the physical delivery method is electroporation. These delivery systems are described in further detail below.

In some forms, the delivery vector carrier is a chemical carrier. Chemical vectors are broadly classified into inorganic particles, lipid based, polymer based, and peptide based. When used, the chemical vector is designed to target specific cells and increase the delivery of genetic material to cytosol or nucleus. Non-limiting examples of suitable chemical non-viral nucleic acid delivery systems include but are not limited to liposome-based nanoparticles such as cationic lipid e.g., DOTAP, neutral phospholipid e.g., DOPE, and cholesterol, solid-lipid nanoparticles such as polysaccharides e.g., hyaluronic acid; niosome nanoparticles; polymer-based nanoparticles such as poly-L-lysin, and lipopeptide-based nanoparticles such as ECO nanoparticles.

The CRISPR-Cas expression vector can further include one or more transcriptional mediation sequences to facilitate formation of a CRISPR complex at one or more target sites. For example, in some forms, a Cas protein and a sgRNA sequence could each be operably linked to separate regulatory elements on separate vectors. In some forms, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. Exemplary transcriptional mediation sequences include nuclear localization sequences, promoter sequences, enhancer sequences, marker sequences, termination signal sequences, polyadenylation signal sequences, and splicing signal sequences. Transcriptional mediation sequences are described in further detail below. In some forms, a single promoter drives expression of a transcript encoding a Cas9 protein and one or more of the sgRNA sequences. Preferably, the promoter used in the CRISPR-Cas expression vector is a U6 promoter, which is active in the cell and facilitate continuous transcription in the target gene. In some forms, the CRISPR-Cas expression vector includes one or more selectable markers for identifying and isolating transfected or transduced cells. In some forms, when the CRISPR-Cas expression vector is directly injected into the liver cells, the CRISPR-Cas expression vector does not include a selectable marker. Only liver cells that take up the plasmids can overgrow and form tumors.

### 2. Transposon Expression Vector

The model generating system typically includes a transposon expression vector to enable expression of one or more oncogenes in the target liver cells. The transposon expression vector includes one or more nucleic acid sequences encoding one or more oncogenes, a pair of inverted terminal repeat (ITR) sequences (also terminal inverted repeats or TIRs), and an antibiotic resistance gene for the selection of cells containing the transposon expression vector.

The transposon expression vector exploits transposon technology to enable expression of one or more oncogenes in the target liver cells. Transposons are segments of DNA with the ability to change their positions within the genome. The most prominent mechanism of transposon movement is “cut-and-paste” transposition, during which a transposase enzyme mediates the excision of the element from its donor location and its reintegration into a new chromosomal locus. During transposition, the transposase (i) interacts with its binding sites in the terminal inverted repeats (TIRs) that define the boundaries of the transposon, (ii) promotes the assembly of a synaptic complex also called paired-end complex (PEC), (iii) catalyzes the excision of the element out of its donor site, and (iv) integrates the excised transposon into a new location in target DNA. The majority of known transposases and retroviral integrases contain a highly conserved triad of amino acids, known as the aspartate-aspartate-glutamate, in short, the DDE (or a variant of it composed of DDD), signature in their C-terminal catalytic domains (Kulkosky et al., Mol. Cell Biol., 12:2331-2338 (1992); Doak et al., Proc. Natl. Acad. Sci. USA, 91:942-946 (1994)). These amino acids play an essential role by coordinating, in general, two Mg⁺⁺ ions required for the catalytic steps (DNA cleavage and joining) of transposition (Bujacz et al., J. Biol. Chem., 272:18161-18168 (1997); Goldgur et al., 95:9150-9154 (1998)). In nature, DNA transposons exist as single, mobile units where the transposase coding sequences are situated between the TIRs. However, the components needed for transposition (transposase gene and ITRs) can be separated and supplied in a trans-arrangement. By constructing an artificial transposon replacing the transposase open reading frame between the TIRs with a sequence of interest and introducing it into a cell along with the transposase encoded on a separate expression plasmid, as messenger RNA or as recombinant protein, the transposase is able to stably integrate the target sequence of interest into the cell's genome enabling sustained transgene expression. Transposons and mechanisms of transposition are described in further detail in the following published reviews: Sandoval-Villegas et al., Int J Mol Sci., 22(10):5084 (2021); Muñoz-López and Garcia-Pérez, Curr Genomics, 11(2):115-28 (2010).

The disclosed transposon expression vector may include nucleic acids encoding a wide variety of genes. In preferred forms, the transposon expression vector includes one or more nucleic acid sequences encoding one or more oncogenes. An exemplary oncogene is cellular myelocytomatosis (c-myc). In some forms, the c-MYC sequence is SEQ ID NO:2. In some forms, the c-MYC sequence is a variant of SEQ ID NO:2. Variant c-MYC sequences include sequences that differ by one or more nucleotide substitutions, additions, or deletions, such as allelic variants. In some forms, the c-MYC sequence is at least 70%, 80%, 85%, 90%, 95 identical to SEQ ID NO:2. An c-MYC sequence that is 97%, 98%, or 99% identical to SEQ ID NO:2 is also contemplated.

c-MYC (Myc sequence):

(SEQ ID NO: 2)
ATGCCCCTCAACGTTAGCTTCACCAACAGGAACTATGACCTCGACTACG
ACTCGGTGCAGCCGTATTTCTACTGCGACGAGGAGGAGAACTTCTACCA
GCAGCAGCAGCAGAGCGAGCTGCAGCCCCCGGCGCCCAGCGAGGATATC
TGGAAGAAATTCGAGCTGCTGCCCACCCCGCCCCTGTCCCCTAGCCGCC
GCTCCGGGCTCTGCTCGCCCTCCTACGTTGCGGTCACACCCTTCTCCCT
TCGGGGAGACAACGACGGCGGTGGCGGGAGCTTCTCCACGGCCGACCAG
CTGGAGATGGTGACCGAGCTGCTGGGAGGAGACATGGTGAACCAGAGTT
TCATCTGCGACCCGGACGACGAGACCTTCATCAAAAACATCATCATCCA
GGACTGTATGTGGAGCGGCTTCTCGGCCGCCGCCAAGCTCGTCTCAGAG
AAGCTGGCCTCCTACCAGGCTGCGCGCAAAGACAGCGGCAGCCCGAACC
CCGCCCGCGGCCACAGCGTCTGCTCCACCTCCAGCTTGTACCTGCAGGA
TCTGAGCGCCGCCGCCTCAGAGTGCATCGACCCCTCGGTGGTCTTCCCC
TACCCTCTCAACGACAGCAGCTCGCCCAAGTCCTGCGCCTCGCAAGACT
CCAGCGCCTTCTCTCCGTCCTCGGATTCTCTGCTCTCCTCGACGGAGTC
CTCCCCGCAGGGCAGCCCCGAGCCCCTGGTGCTCCATGAGGAGACACCG
CCCACCACCAGCAGCGACTCTGAGGAGGAACAAGAAGATGAGGAAGAAA
TCGATGTTGTTTCTGTGGAAAAGAGGCAGGCTCCTGGCAAAAGGTCAGA
GTCTGGATCACCTTCTGCTGGAGGCCACAGCAAACCTCCTCACAGCCCA
CTGGTCCTCAAGAGGTGCCACGTCTCCACACATCAGCACAACTACGCAG
CGCCTCCCTCCACTCGGAAGGACTATCCTGCTGCCAAGAGGGTCAAGTT
GGACAGTGTCAGAGTCCTGAGACAGATCAGCAACAACCGAAAATGCACC
AGCCCCAGGTCCTCGGACACCGAGGAGAATGTCAAGAGGCGAACACACA
ACGTCTTGGAGCGCCAGAGGAGGAACGAGCTAAAACGGAGCTTTTTTGC
CCTGCGTGACCAGATCCCGGAGTTGGAAAACAATGAAAAGGCCCCCAAG
GTAGTTATCCTTAAAAAAGCCACAGCATACATCCTGTCCGTCCAAGCAG
AGGAGCAAAAGCTCATTTCTGAAGAGGACTTGTTGCGGAAACGACGAGA
ACAGTTGAAACACAAACTTGAACAGCTACGGAACTCTTGTGCGGACCCA
GCTTTCTTGTACAAAGTGGTTGATATCCAGCACAGTGGCGGCCGCTCGA
GTCTAGAGGGCCCGCGGTTCGAAGGTAAGCCTATCCCTAACCCTCTCCT
CGGTCTCGATTCTACGCGTACCGGTTAG.

In some forms, the target oncogene is one or more of TERT (telomerase reverse transcriptase), RAS (Rat sarcoma), and/or CTNNB1 (Catenin (Cadherin-Associated Protein), Beta 1). In some forms, the sequence for the target oncogene is derived from a human or mouse TERT, RAS, or CTNNB1 sequence. In some forms, the TERT sequence is at least 70%, 80%, 85%, 90%, 95 identical to a canonical TERT, RAS, or CTNNB1 sequence.

In some forms, the nucleic acid encoding the oncogene may include a sequence of bases that is endogenous and/or exogenous to target cells. An exogenous sequence is one that is not present in the target cell, while an endogenous sequence is one that pre-exists in the target cell prior to delivery of the transposon expression vector. Either way, the nucleic acid encoding the oncogene is exogenous to the target cell, since it originates from a source other than the target cell and is introduced into the target cell by the methods described below. In some forms, the exogenous nucleic acid may be an oncogene whose protein product is not well characterized. In such forms, the transposon expression vector is employed to stably introduce the oncogene into the target cell and observe changes in the cell phenotype to characterize the oncogene. In some forms, the exogenous nucleic acid encodes a protein of interest which is to be produced by the cell. The nucleic acid encoding the oncogene may vary in size. The upper and lower limits of the size of the nucleic acid encoding the oncogene may readily be determined empirically by those of skill in the art.

The transposon expression vector includes a pair of inverted terminal repeat sequences (also referred terminal inverted repeats or TIRs) located on either side of the sequence encoding the oncogene. Generally, the ITR sequences contain DNA-binding sites that are recognized by the transposase to form the synaptic protein-DNA complex, thus facilitating the cleavage at both strands and the subsequent transposon release. Depending on the transposon family, ITR sequences can vary in sequence length and TR-binding site numbers and patterns. As a first approximation, ITR sequences can often be divided in two functional modules or domains (Szab6 et al., 2010; Craig et al., 2002). The first domain includes two or three terminal base pairs and is involved in DNA cleavage and strand transfer reactions. The second domain is an internal sequence in the ITR and is required for TR specific recognition and binding.

ITR sequences from various transposon superfamilies can be incorporated in the transposon expression vector. Suitable transposon superfamilies include but are not limited to Tc1/mariner, PIF/Harbinger, hAT, Mutator, Merlin, Transib, P, piggyBac or CACTA. Exemplary ITR sequences include but are not limited to Sleeping Beauty, piggyBac, Tn5, Tn7, and Mu ITR sequences. In preferred forms, the ITR sequences are derived from the Tc1/mariner superfamily. In preferred forms, the pair of ITR sequences are Sleeping Beauty (SB) ITR sequences. The Sleeping Beauty (SB) transposon system and the mechanism thereof is further described in the following published reviews: Ivics Z, et al., Cell, 91:501-510 (1997); Grabundzija et al., Nucl. Acids Res., 41(3):1829-1847 (2012); Sumiyoshi et al., Human Gene Therapy, 20(12):1607-1626 (2009); Huang, et al., Molecular Therapy, 18(10):1803-1813 (2010); and Hackett et al., Genome Biol., 8:S12 (2007).

Exemplary SB ITR sequences include SEQ ID NO:8 and SEQ ID NO:9 (WO 1998/040510) and SEQ ID NO:25. In preferred forms, the ITR sequence includes SEQ ID NO:25.

SEQ ID NO: 8 (5′ to 3′):
AGTTGAAGTCGGAAGTTTACATACACTTAAGTTGGAGTCATTAAAACTC
GTTTTTCAACTACACCACAAATTTCTTGTTAACAAACAATAGTTTTGGC
AAGTCAGTTAGGACATCTACTTTGTGCATGACACAAGTCATTTTTCCAA
CAATTGTTTACAGACAGATTATTTCACTTATAATTCACTGTATCACAAT
TCCAGTGGGTCAGAAGTTTACATACACTAA
SEQ ID NO: 9 (5′ to 3′):
TTGAGTGTATGTTAACTTCTGACCCACTGGGAATGTGATGAAAGAAATA
AAAGCTGAAATGAATCATTCTCTCTACTATTATTCTGATATTTCACATT
CTTAAAATAAAGTGGTGATCCTAACTGACCTTAAGACAGGGAATCTTTA
CTCGGATTAAATGTCAGGAATTGTGAAAAAGTGAGTTTAATGTATTTGG
CTAAGGTGTATGTAAACTTCCGACTTCAACTG
SEQ ID NO: 25 (5′ to 3′):
TTGGAGTCATTAAAACTCGTTTTTCAACTACTCCACAAATTTCTTGTTA
ACAAACAATAGTTTTGGCAAGTCAGTTAGGACATCTACTTTGTGCATGA
CACAAGTCATTTTTCCAACAATTGTTTACAGACAGATTATTTCACTTAT
AATTCACTGTATCACAAT

In some forms, each one of the pair of ITR sequences includes one direct repeat. In some forms, each one of the pair of ITR sequences includes two direct repeats in each inverted repeat sequence. Exemplary direct repeat sequences include but are not limited SEQ ID NOs:4-7, SEQ ID NO:26, and SEQ ID NO:27. In preferred forms, the direct repeat sequences include SEQ ID NO:26 for the 5′ or 3′ outer direct repeat and SEQ ID NO:27 for the 5′ or 3′ inner direct repeat.

The 5′ outer repeat:

(SEQ ID NO: 32)
5′-GTTCAAGTCGGAAGTTTACATACACTTAG-3′;
The 5′ inner repeat:
(SEQ ID NO: 5)
5′-CAGTGGGTCAGAAGTTTACATACACTAAGG-3′;
The 3′ inner repeat:
(SEQ ID NO: 6)
5′-CAGTGGGTCAGAAGTTAACATACACTCAATT-3′;
The 3′ outer repeat:
(SEQ ID NO: 7)
5′-AGTTGAATCGGAAGTTTACATACACCTTAG-3′;
The 5′ or 3′ outer direct repeat:
(SEQ ID NO: 26)
5′-CAGTTGAAGTCGGAAGTTTACATACACTTAAG-3′;
The 5′ or 3′ inner direct repeat:
(SEQ ID NO: 27)
5′-TCCAGTGGGTCAGAAGTTTACATACACTAAGT-3′.

ITR sequences and direct repeat sequences are further described in U.S. Pat. No. 6,613,752; U.S. Patent Publication No. 2005/0003542, and U.S. Patent Publication No. 2006/0252140, all of which are herein incorporated by reference in their entireties.

In some forms, the inverted repeat domain can include one or more restriction endonuclease recognized sites, e.g., restriction sites. These restriction sites may be located between the flanking inverted repeats, which serves as a site for insertion of the nucleic acid encoding the oncogene or another gene of interest. The restriction sites are recognized by restriction enzymes which cleaves the DNA sequences into fragments at or near the specific sequence specific sites within the restriction sites. A variety of restriction sites and restriction enzymes are known in the art. Exemplary restriction enzymes include but are not limited to HindIII, PstI, SalI, AccI, HincII, XbaI, BamHI, SmaI, XmaI, KpnI, SacI, and EcoRI. In some forms, the transposon expression vector includes a polylinker, i.e., a closely arranged series or array of sites recognized by a plurality of different restriction enzymes, such as those listed above.

In some forms, the transposon expression vector includes an antibiotic resistance gene for the selection of cells containing the transposon expression vector. Exemplary antibiotic resistance genes that can be used include kanamycin (kan^{r}), spectinomycin, streptomycin, ampicillin (amp^{r}), carbenicillin (cm^{r}), bleomycin, erythrmycin, polymyxin B, tetracycline (tet^{r}), and chloramphenicol. Selectable marker genes are also contemplated as alternatives for the selection of cells containing the transposon expression vector. Suitable selectable marker genes include but are not limited to the thymidine kinase gene, the dihydrofolate reductase gene, the xanthine-guanine phosphoribosyl transferase gene, CAD, the adenosine deaminase gene, the asparagine synthetase gene, the hygromycin B phosphotransferase gene, and genes whose expression provides for the presence of a detectable product, either directly or indirectly, e.g., β-galactosidase, GFP. Typically, the antibiotic resistance gene or selection marker used for the selection of cells containing the transposon expression vector is different from that used for selection of cells containing the transposase expression vector.

In preferred forms, in vitro expansion is not required to select the cells. Thus, in preferred forms, the transposon expression vector does not include an antibiotic resistance gene, in part because the cancer cells that take up the genome-editing plasmids will outgrow the non-cancer cells.

The transposon expression vector further includes a regulatory element for controlling the expression of the oncogene in a host cell, wherein the regulatory element is selected from the group consisting of promoters, enhancers, silencers, and insulators. In some forms, the promoter is a liver-specific promoter. Exemplary liver-specific promoters include albumin promoter, the alpha-1 antitrypsin promoter, the apolipoprotein E promoter, alpha-fetoprotein (AFP) promoter, CYP1A1 promoter, CYP2B6 promoter, CYP3A4 promoter, fatty acid-binding protein (FABP) promoter, glucose-6-phosphatase (G6Pase) promoter, Hepatitis B virus (HBV) core promoter, HNF1-alpha promoter, HNF4-alpha promoter, Interleukin-6 (IL-6) promoter, and Transferrin promoter.

In preferred forms, when the transposon expression vector e.g., plasmid, is injected directly into liver cells, the transposon expression vector does not include a liver-specific promoter. In some forms, the promoter in the transposon vector is selected from the group containing T7 promoter, CMV promoter, SV40 promoter, and EF1α promoter. In preferred forms, the promoter included on the transposon expression vector is an EF1α promoter.

In some forms, the transposon expression vector further includes a reporter gene sequence located within the ITR sequence for monitoring the expression of the oncogene in a host cell. Suitable proteins encoded by the reporter gene sequence includes but are not limited to green fluorescent protein (GFP), red fluorescent protein (RFP), luciferase, and β-galactosidase.

### 3. Transposase Expression Vector

The model generating system typically contains a transposase expression vector designed to facilitate expression of the transposase in the target liver cells. The transposase expression vector includes a nucleic acid sequence encoding a transposase and a first antibiotic resistance gene for the selection of cells containing the transposase expression vector.

A transposase is any of a class of enzymes capable of binding to the end of a transposon and catalyzing its movement to another part of a genome, typically by a cut-and-paste mechanism or a replicative mechanism, in a process known as transposition (reviewed in Hickman and Dyda, Microbiol Spectr., 3(2):MDNA3-0034-2014 (2015); Sandoval-Villegas et al., Int J Mol Sci., 22(10):5084 (2021)). In some forms, the transposase binds to any part of the DNA molecule, wherein the target site is located at any position. In some forms, the transposase binds to specific sequences on the DNA molecule. The transposase then cuts the target site to produce sticky ends, releases the transposon and ligates it into the target site. More details of the mechanisms of transposase enzymes are described above in Section II(2) titled “Transposon Expression Vector.”

Suitable transposases that can be included in the transposase expression vector include but are not limited to Sleeping Beauty, piggyBac, Tn5, Tn7, and Mu. In preferred forms, the nucleic acid sequence included in the transposase expression vector encodes a sleeping Beauty transposase, piggyBac transposase, or Tol2 transposase. More preferably, the nucleic acid included in the transposase expression vector encodes a Sleeping Beauty transposase. The structure of the SB transposase is described in Ochmann and Ivics, Viruses, 13(1):76 (2021). Briefly, the SB transposase is a 340 amino acid (aa) protein composed of an N-terminal DNA-binding domain (DBD) (aa 1-109) with two subdomains called PAI and RED, followed by a flexible interdomain linker (aa 110-127) carrying a nuclear localization signal (NLS) that overlaps with the DBD (aa 104-120) and a C-terminal catalytic domain (aa 128-340) harboring the DDE triad (D153, D244, E279) (Ivics, et al., Cell, 91:501-510 (1997)).

In some forms, the Sleeping Beauty (SB) transposase is SB10 transposase or a variant thereof. SB10 transposase is considered the first functional version of SB transposases constructed by fusing portions of two inactive transposon sequences from Atlantic salmon (Salmo salar) and one inactive transposon sequence from rainbow trout (Oncorhynchus mykiss) and then repairing small deficits in the functional domains of the transposase enzyme. The amino acid sequence of the wild-type SB transposase is represented by SEQ ID NO:10. In some forms, the SB transposase is a variant having about 70%, about 75%, about 80%, about 90%, or about 95% sequence identity to SEQ ID NO:10. Enhanced variants of SB10 transposase is described in U.S. Pat. No. 7,985,739, which is incorporated herein in its entirety.

SB10 or originally derived transposase (NCBI Accession ID: AEL94441.1):

(SEQ ID NO: 10)
MGKSKEISQDLRKKIVDLHKSGSSLGAISKRLKVPRSSVQTIVRKYKHH
GTTQPSYRSGRRRVLSPRDERTLVRKVQINPRTTAKDLVKMLEETGTKV
SISTVKRVLYRHNLKGRSARKKPLLQNRHKKARLRFATAHGDKDRTFWR
NVLWSDETKIELFGHNDHRYVWRKKGEACKPKNTIPTVKHGGGSIMLWC
GFAAGGTGALHKIDGIMRKENYVDILKQHLKTSVRKLKLGRKWVFQMDN
DPKHTSKVVAKWLKDNKVKVLEWPSQSPDLNPIENLWAELKKRVRARRP
TNLTQLHQLCQEEWAKIHPTYCGKLVEGYPKRLTQVKQFKGNATKY.

The nucleic acid sequence for SB10 transposase (addgene #24551) is provided below (5′ to 3′):

(SEQ ID NO: 28)
ATGGGAAAATCAAAAGAAATCAGCCAAGACCTCAGAAAAAAAATTGTAG
ACCTCCACAAGTCTGGTTCATCCTTGGGAGCAATTTCCAAACGCCTGAA
AGTACCACGTTCATCTGTACAAACAATAGTACGCAAGTATAAACACCAT
GGGACCACGCAGCCGTCATACCGCTCAGGAAGGAGACGCGTTCTGTCTC
CTAGAGATGAACGTACTTTGGTGCGAAAAGTGCAAATCAATCCCAGAAC
AACAGCAAAGGACCTTGTGAAGATGCTGGAGGAAACAGGTACAAAAGTA
TCTATATCCACAGTAAAACGAGTCCTATATCGACATAACCTGAAAGGCC
GCTCAGCAAGGAAGAAGCCACTGCTCCAAAACCGACATAAGAAAGCCAG
ACTACGGTTTGCAACTGCACATGGGGACAAAGATCGTACTTTTTGGAGA
AATGTCCTCTGGTCTGATGAAACAAAAATAGAACTGTTTGGCCATAATG
ACCATCGTTATGTTTGGAGGAAGAAGGGGGAGGCTTGCAAGCCGAAGAA
CACCATCCCAACCGTGAAGCACGGGGGTGGCAGCATCATGTTGTGGGGG
TGCTTTGCTGCAGGAGGGACTGGTGCACTTCACAAAATAGATGGCATCA
TGAGGAAGGAAAATTATGTGGATATATTGAAGCAACATCTCAAGACATC
AGTCAGGAAGTTAAAGCTTGGTCGCAAATGGGTCTTCCAAATGGACAAT
GACCCCAAGCATACTTCCAAAGTTGTGGCAAAATGGCTTAAGGACAACA
AAGTCAAGGTATTGGAGTGGCCATCACAAAGCCCTGACCTCAATCCTAT
AGAAAATTTGTGGGCAGAACTGAAAAAGCGTGTGCGAGCAAGGAGGCCT
ACAAACCTGACTCAGTTACACCAGCTCTGTCAGGAGGAATGGGCCAAAA
TTCACCCAACTTATTGTGGGAAGCTTGTGGAAGGCTACCCGAAACGTTT
GACCCAAGTTAAACAATTTAAAGGCAATGCTACCAAATACTAG.

In some forms, the Sleeping Beauty (SB) transposase is SB13 transposase or a variant thereof. The amino acid sequence of the wild-type SB transposase is represented by SEQ ID NO:30. In some forms, the SB transposase is a variant having about 70%, about 75%, about 80%, about 90%, or about 95% sequence identity to SEQ ID NO:30.

SB13 transposase:

(SEQ ID NO: 30)
MGKSKEISQDLRAKIVDLHKSGSSLGAISKRLAVPRSSVQTIVRKYKHH
GTTQPSYRSGRRRVLSPRDERTLVRKVQINPRTAAKDLVKMLEETGTKV
SISTVKRVLYRHNLKGRSARKKPLLQNRHKKARLRFATAHGDKDRTFWR
NVLWSDETKIELFGHNDHRYVWRKKGEACKPKNTIPTVKHGGGSIMLWG
CFAAGGTGALHKIDGIMRKENYVDILKQHLKTSVRKLKLGRKWVFQMDN
DPKHTSKVVAKWLKDNKVKVLEWPSQSPDLNPIENLWAELKKRVRARRP
TNLTQLHQLCQEEWAKIHPTYCGKLVEGYPKRLTQVKQFKGNATKY.

The nucleic acid sequence for SB13 transposase is provided below (5′ to 3′):

(SEQ ID NO: 31)
ATGGGAAAATCAAAAGAAATCAGCCAAGACCTCAGAGCGAAAATTGTAG
ACCTCCACAAGTCTGGTTCATCCTTGGGAGCAATTTCCAAACGCCTGGC
GGTACCACGTTCATCTGTACAAACAATAGTACGCAAGTATAAACACCAT
GGGACCACGCAGCCGTCATACCGCTCAGGAAGGAGACGCGTTCTGTCTC
CTAGAGATGAACGTACTTTGGTGCGAAAAGTGCAAATCAATCCCAGAAC
AGCGGCAAAGGACCTTGTGAAGATGCTGGAGGAAACAGGCACAAAAGTA
TCTATATCCACAGTAAAACGAGTCCTATATCGACATAACCTGAAAGGCC
GCTCAGCAAGGAAGAAGCCACTGCTCCAAAACCGACATAAGAAAGCCAG
ACTACGGTTTGCAACTGCACATGGGGACAAAGATCGTACTTTTTGGAGA
AATGTCCTCTGGTCTGATGAAACAAAAATAGAACTGTTTGGTCATAATG
ACCATCGTTATGTTTGGAGGAAGAAGGGGGAGGCTTGCAAGCCGAAGAA
CACCATCCCAACCGTGAAGCACGGGGGTGGCAGCATCATGTTGTGGGGG
TGCTTTGCCGCAGGAGGGACTGGTGCACTTCACAAAATAGATGGCATCA
TGAGGAAGGAAAATTATGTGGATATATTGAAGCAACATCTCAAGACATC
AGTCAGGAAGTTAAAGCTTGGTCGCAAATGGGTCTTCCAAATGGACAAT
GACCCCAAGCATACTTCCAAAGTTGTGGCAAAATGGCTTAAGGACAACA
AAGTCAAGGTATTGGAGTGGCCATCACAAAGCCCTGACCTCAATCCTAT
AGAAAATTTGTGGGCAGAACTGAAAAAGCGTGTGCGAGCAAGGAGGCCT
ACAAACCTGACTCAGTTACACCAGCTCTGTCAGGAGGAATGGGCCAAAA
TTCACCCAACTTATTGTGGGAAGCTTGTGGAAGGCTACCCGAAACGTTT
GACCCAAGTTAAACAATTTAAAGGCAATGCTACCAAATACTAG.

In some forms, the transposase is a hyperactive variant of a transposase. A “hyperactive” transposase is a transposase that is more active than the naturally occurring transposase from which it is derived. “Hyperactive” transposases are thus not naturally occurring sequences. In some forms, the hyperactive transposase can be a hyperactive Sleeping Beauty transposase (reviewed in Hudecek et al, Critical Reviews in Biochemistry and Molecular Biology, 52(4):355-380 (2017); Amberger and Ivics, BioEssays, 41(11) (2020)). Briefly, hyperactive transposases contain amino acid substitutions spanning almost the entire SB transposase polypeptide, thereby eliciting changes in catalytic activity. These amino acid replacements were conducted either by systematic alanine-scanning (Wang et al., 2016b; Yant, et al., Mol. Cell Biol. 24:9239-9247 (2004), by “transplanting” single amino acids or small (2-7 amino acid) blocks of amino acids from related transposases (Baus et al., Mol. Therapy, 12:1148-1156 (2005); Geurts et al., Mol. Ther., 8:108-117 (2003); Zayed et al., 2004), and by replacement of selected amino acid residues based on charge (Zayed et al., 2004). For example, hyperactive SB transposases in which activity is increased by the systematic exchange of the N-terminal 95AA of the SB transposase for alanine is described (Yant, et al., Mol. Cell Biol. 24:9239-9247 (2004)). A second-generation SB transposase called SB11 contains five amino acid replacements (selected based on a phylogenetic comparison to active Tc1/mariner transposases) over the first-generation transposase and is about 3-fold more active than the first-generation SB transposase (Geurts et al., Mol. Ther., 8:108-117 (2003). Another example of a hyperactive SB transposase is SB16 which combines five individual hyperactive mutations and is reported to have a 16-fold activity increase as compared to natural SB10 (Baus et al., Mol. Therapy, 12:1148-1156 (2005). SB100X is another example of a hyperactive SB transposase reported to demonstrate ˜100-fold enhancement in efficiency when compared to first-generation transposases, allowing reduced transposase quantity without compromising high transposition rates (Mates et al., Nat Genet., 41(6):753-61 (2009); Jin et al., Gene Ther., 18(9):849-856). Exemplary amino acid sequences for SB100X transposases are represented by SEQ ID NO:11 and SEQ ID NO:12. Additional hyperactive variants of SB transposases that can be used are described in U.S. Pat. No. 9,228,180, which is incorporated herein in its entirety.

SB100X Transposase (NCBI Accession ID: 5CR4_A):

(SEQ ID NO: 11)
GPSGHSARKKPLLQNRHKKARLRFATAHGDKDRTFWRNVLWSDETKIEL
FGHNDHRYVWRKKGEACKPKNTIPTVKHGGGSIMLWGCFAAGGTGALHK
IDGIMDAVQYVDILKQHLKTSVRKLKLGRKWVFQHDNDPKHTSKVVAKW
LKDNKVKVLEWPSQSPDLNPIENLWAELKKRVRARRPTNLTQLHQLCQE
EWAKIHPNYCGKLVEGYPKRLTQVKQFKGNATKY.

SB100X Transposase (NCBI Accession ID: 5CR4_B):

(SEQ ID NO: 12)
GPSGHSARKKPLLQNRHKKARLRFATAHGDKDRTFWRNVLWSDETKIEL
FGHNDHRYVWRKKGEACKPKNTIPTVKHGGGSIMLWGCFAAGGTGALHK
IDGIMDAVQYVDILKQHLKTSVRKLKLGRKWVFQHDNDPKHTSKVVAKW
LKDNKVKVLEWPSQSPDLNPIENLWAELKKRVRARRPTNLTQLHQLCQE
EWAKIHPNYCGKLVEGYPKRLTQVKQFKGNATKY.

In some forms, the SB100X transposase has the following nucleic acid sequence (addgene #127909) (5′ to 3′):

(SEQ ID NO: 29)
ATGGGAAAATCAAAAGAAATCAGCCAAGACCTCAGAAAAAGAATTGTAG
ACCTCCACAAGTCTGGTTCATCCTTGGGAGCAATTTCCAAACGCCTGGC
GGTACCACGTTCATCTGTACAAACAATAGTACGCAAGTATAAACACCAT
GGGACCACGCAGCCGTCATACCGCTCAGGAAGGAGACGCGTTCTGTCTC
CTAGAGATGAACGTACTTTGGTGCGAAAAGTGCAAATCAATCCCAGAAC
AACAGCAAAGGACCTTGTGAAGATGCTGGAGGAAACAGGTACAAAAGTA
TCTATATCCACAGTAAAACGAGTCCTATATCGACATAACCTGAAAGGCC
ACTCAGCAAGGAAGAAGCCACTGCTCCAAAACCGACATAAGAAAGCCAG
ACTACGGTTTGCAACTGCACATGGGGACAAAGATCGTACTTTTTGGAGA
AATGTCCTCTGGTCTGATGAAACAAAAATAGAACTGTTTGGCCATAATG
ACCATCGTTATGTTTGGAGGAAGAAGGGGGAGGCTTGCAAGCCGAAGAA
CACCATCCCAACCGTGAAGCACGGGGGTGGCAGCATCATGTTGTGGGGG
TGCTTTGCTGCAGGAGGGACTGGTGCACTTCACAAAATAGATGGCATCA
TGGACGCGGTGCAGTATGTGGATATATTGAAGCAACATCTCAAGACATC
AGTCAGGAAGTTAAAGCTTGGTCGCAAATGGGTCTTCCAACACGACAAT
GACCCCAAGCATACTTCCAAAGTTGTGGCAAAATGGCTTAAGGACAACA
AAGTCAAGGTATTGGAGTGGCCATCACAAAGCCCTGACCTCAATCCTAT
AGAAAATTTGTGGGCAGAACTGAAAAAGCGTGTGCGAGCAAGGAGGCCT
ACAAACCTGACTCAGTTACACCAGCTCTGTCAGGAGGAATGGGCCAAAA
TTCACCCAAATTATTGTGGGAAGCTTGTGGAAGGCTACCCGAAACGTTT
GACCCAAGTTAAACAATTTAAAGGCAATGCTACCAAATACTAG.

In some forms, the transposase expression vector includes a first antibiotic resistance gene for the selection of cells containing the transposase expression vector. Exemplary antibiotic resistance genes that can be used include kanamycin (kan^{r}), spectinomycin, streptomycin, ampicillin (amp^{r}), carbenicillin (cm^{r}), bleomycin, erythrmycin, polymyxin B, tetracycline (tet^{r}), and chloramphenicol. In preferred forms, the preparation of the transposase expression vector does not require clonal selection. Thus, in preferred forms, the transposase expression vector does not include an antibiotic resistance gene.

Selectable marker genes are also contemplated as alternatives selection of cells containing the transposase expression vector. Suitable selectable marker genes include but are not limited to the thymidine kinase gene, the dihydrofolate reductase gene, the xanthine-guanine phosphoribosyl transferase gene, CAD, the adenosine deaminase gene, the asparagine synthetase gene, the hygromycin B phosphotransferase gene, and genes whose expression provides for the presence of a detectable product, either directly or indirectly, e.g., β-galactosidase, GFP.

The transposase can be delivered into the target liver cells as a protein or as ribonucleic acid, including mRNA, as DNA, e.g., as extrachromosomal DNA including, but not limited to, episomal DNA, as plasmid DNA, or as viral nucleic acid. Furthermore, the transposase can be delivered into the target liver cells via a nucleic acid vector such as a plasmid, or as a gene expression vector, including a viral vector. Therefore, the nucleic acid can be circular or linear. Exemplary delivery vectors include DNA plasmid, minicircles, minimized DNA vector, mRNA, or recombinant protein. In preferred forms, the transposase is delivered using a plasmid vector. In this form, the transposase is included in an expression cassette and incorporated into a plasmid along with necessary components for propagation and manufacturing in bacterial cells such as an origin of replication and an antibiotic resistance gene. In some forms, the transposase expression plasmid can be electroporated into target liver cells.

In some forms, the transposase can be delivered into the target liver cells via minicircles or minimized DNA vectors. DNA minicircles are circular expression cassettes derived from a parent plasmid, containing almost exclusively the “gene of interest” and its regulating sequence motifs and devoid of any bacterial plasmid DNA backbone. Minivectors are minimized, non-viral DNA vectors similar to minicircles but with some important differences. Like minicircles, minivectors are synthesized from a parent plasmid via site-specific recombination. Encoding only the genetic payload and short integration sequences, minivectors can be engineered as small as ˜350 bp and generated in high yields. Exemplary minimized DNA vectors include but are not limited to plasmid-free of antibiotic resistance markers (pFAR), minicircle (MC), and doggybone DNA (dbDNA). Plasmids free of antibiotic resistance genes (pFAR) can be produced from engineered E. coli that bear a nonsense mutation in the essential thyA thymidylate synthase gene (Vandermeulen, et al., J. Gene Med, 12:323-332 (2010)). pFAR vectors encoding a nonsense suppressor tRNA are then able to alter the reading and restore bacterial growth. In addition to avoiding the presence of antibiotic resistance genes, pFARs greatly reduce the overall length of the construct and have shown lower toxicities and efficient transgene delivery in association with the SB transposon system (Garcia-Garcia, et al., Mol. Ther. Nucleic Acids, 9:1-11 (2017); Hernandez et al., Mol. Ther. Methods Clin. Dev., 15:403-417 (2019)).

In some forms, the transposase can be delivered to the target cell as in vitro-transcribed messenger RNA (mRNA). This form is suitable for short-term expression, thereby reducing toxicity and avoiding risks of chromosomal integration. For example, Stabilized Non-Immunogenic Messenger RNA (SNIM-RNA) is currently being used with SB100X, and involves mRNAs bearing chemical modifications for increased stability and lower activation of the innate immune response associated with in vitro-transcribed mRNA (Holstein et al., Mol. Ther. 26:1137-1153 (2018)). In other forms, the transposase can be directly delivered to the target cell as a recombinant protein. For example, studies with SB transposases (Jarver, et al., nt. J. Pept. Res. Ther., 14:58-63 (2008) and PB transposases (Lee et al, Biomaterials, 32:6264-6276 (2011) linked to cell-penetrating peptides (CPPs) have reported that transposases could be delivered as proteins and penetrate cellular membranes along with the transposon.

In preferred forms, the nucleic acid sequence encoding the SB transposase and the nucleic acid encoding the SB ITRs are present on separate vectors, e.g., separate plasmids. However, in some forms, the transposase encoding domain may be present on the same vector as the SB ITR sequences, e.g., on the same plasmid. When present on the same vector, the SB transposase encoding region or domain is located outside the inverted repeat flanked domain. In other words, the transposase encoding region is located external to the region flanked by the inverted repeats, i.e., outside the inverted repeat domain. Put another way, the transposase encoding region is positioned to the left of the left terminal inverted repeat or the right of the right terminal inverted repeat.

### 4. Expression Elements

Each one of the components of the model generating system, i.e., the CRISPR-Cas expression vector, transposon expression vector, and the transposase expression vector, can include one or more of a variety of expression elements to modify expression of the genes of interest in the target cells whose genome is modified by integration of the model generating system. Suitable expression elements include but are not limited to promoters, enhancers, termination and polyadenylation signal elements, and splicing signal elements.

a. Promoter/Enhancer Sequences

The level of transgene expression in the target cells may be regulated by one or more transcriptional promoter sequences and/or enhancer sequences within the transgene expression cassette. In some forms, combinations of one or more tissue-specific promoters and one or more tissue-specific enhancers, such as liver-specific promoters and enhancers may be used to regulate transgene expression in the target cells.

In some forms, the nucleic acid sequence encodes a tissue-specific promoter e.g., a liver specific promoter. Non-limiting examples of liver-specific promoters include, but are not limited to albumin promoter (Alb), alpha-1-antitrpsin promoter (AAT), apolipoprotein A-I promoter (ApoA1), apolipoprotein E promoter (ApoE), hepatic nuclear 1 alpha promoter (HNF1α), hepatic nuclear 4 alpha promoter (HNF4α), transthyretin promoter (TTR), glucose-6-phosphatase promoter (G6Pase), liver fatty acid-binding protein promoter (L-FABP), c-reactive protein promoter (CRP), glucokinase promoter (GK), liver X receptor alpha promoter (LXRα), phosphoenolpyruvate carboxykinase promoter (PEPCK); retinol-binding protein 4 promoter (RBP4), liver-specific organic anion transporter 2 promoter (OATP2), and cytochrome P450 family promoters e.g. CYP1A2, CYP2E1, CYP2B6, CYP2C9, and CYP3A4. In some forms, these liver-specific promoters are used alone or in combination to achieve liver-specific expression within the target liver cells. Additional promoters such as strong constitutive promoters capable of promoting expression of an associated coding DNA sequence in the liver target cells are also contemplate. Exemplary strong constitutive promoters include but are not limited to the human and murine cytomegalovirus promoter, truncated CMV promoters, human serum albumin promoter (HSA) and the alpha-1-antitrypsin promoter.

In preferred forms, when the expression vector e.g., plasmid, are injected directly into liver cells, the expression vector does not include a liver-specific promoter. In some forms, the promoter in the expression vector is selected from the group containing T7 promoter, CMV promoter, SV40 promoter, and EF1α promoter. In preferred forms, the promoter included on the expression vector is an EF1α promoter.

In some forms, to improve tissue-specific expression, other regulatory elements may be operably linked to the transgene, e.g., the Woodchuck Hepatitis Virus Post-Regulatory Element (WPRE) (Donello et al. (1998) J. Virol, 72: 5085-5092) or the bovine growth hormone (BGH) polyadenylation site.

In some forms, the enhancer element is a tissue-specific enhancer sequence e.g., a nucleic acid encoding liver specific enhancer. Liver-specific enhancers are regulatory elements capable of boosting tissue-specific expression of the target genes in the target liver cells. Liver-specific enhancer elements can be used in combination with liver-specific promoters to fine-tune the expression of the target gene in the target liver cells. Such liver-specific enhancers include one or more human serum albumin enhancers (HSA), human prothrombin enhancers (HPrT), alpha-1 microglobulin enhancers (A1 MB), and intronic aldolase enhancers. Exemplary liver-specific enhancer elements that can be used include but are not limited to albumin enhancer (AlbE), alpha-fetoprotein enhancer (AFPE), apolipoprotein E enhancer (ApoEE), hepatocyte nuclear factor 1α (HNF1αE), hepatocyte nuclear factor 4α (HNF4αE), liver-specific HNF1α/4α response element (LHRE), liver-specific CCAAT/enhancer-binding protein alpha (C/EBPα) enhancer, liver X receptor response element (LXRRE), liver-specific peroxisome proliferator-activated receptor alpha (PPARα) enhancer, liver-specific sterol regulatory element-binding protein (SREBP1) enhancer, liver specific farnesoid X receptor (FXR) enhancer, and liver-specific retinoid X receptor (RXR) enhancer. In some forms, multiple enhancer elements may be combined in order to achieve higher expression e.g., two identical enhancers may be combined with a liver-specific promoter.

b. Selection Marker Sequences

The components of the model generating system can include one or more selection marker sequences. Selection marker sequences are genetic elements that are used to identify and select cells that have incorporated the expression construct or the foreign gene of interest. Cells harboring a component of the model generating system such as the CRISPR-Cas expression vector or the transposon expression vector, may include a marker construct, to enable monitoring of target gene knockdown or overexpression, and the formation and/or progression of HCC tumors.

In some forms, the selection marker sequence encodes a gene which confers resistance to a selective agent e.g., an antibiotic. Exemplary antibiotic resistance genes include but are not limited to ampicillin resistance gene (Amp^{R}), kanamycin resistance gene (KanR), neomycin resistance gene (NeoR), tetracycline resistance genes (Tet^{R}) such as tetA, tetB, tetM, and tetO; chloramphenicol resistance gene (CmR) such as catA1, catA2, and catA3; hygromycin resistance gene (HygR), blasticidin resistance gene (BsdR), puromycin resistance gene (Pac), zeocin resistance gene (Sh ble), phleomycin resistance gene (BleoR), erythromycin resistance gene (ErmR), streptomycin resistance gene (StrR), gentamicin resistance gene (GmR), spectinomycin resistance gene (SpcR), trimethoprim resistance gene (TmpR), rifampicin resistance gene (RifR), β-lactamase (bla) enzymes such as blaTEM, blaSHV, blaCTX-M, and blaNDM; methicillin resistance genes such as mecA and mecC; vancomycin resistance genes such as vanA, vanB, and vanC; erm genes such as ermB, ermC, and ermF; quinolone resistance (qnr) genes such as qnrA, qnrB, and qnrS; sulfonamide (sul) resistance genes such as sull, sul2, and sul3; and dihydrofolate reductase (dfr) enzymes such as dfrA1, dfrA12, and dfrA17.

In some forms, the selection marker is a fluorescent marker, fluorophore or fluorescent dye, molecules that enable the identification of cells by absorbing light at specific wavelengths and emit light at longer wavelengths. Suitable fluorescent markers include but are not limited to green fluorescent protein (GFP), enhanced yellow fluorescent protein (EYFP), enhanced cyan fluorescent protein (ECFP), mCherry, fluorescein isothiocyanate (FITC), tetramethylrhodamine isothiocyanate (TRITC), Texas red, alexa fluor dyes such as Alexa Fluor 488, Alexa Fluor 546, Alexa Fluor 594, and Alexa Fluor 647, DAPI (4′,6-diamidino-2-phenylindole), Renilla Reniformis green fluorescent protein, GFPmut2, GFPuv4, yellow fluorescent protein (YFP), cyan fluorescent protein (CFP), blue fluorescent protein (BFP), enhanced blue fluorescent protein (EBFP), citrine and red fluorescent protein from discosoma (dsRED), orange fluorescent proteins such as mOrange and mOrange2, mApple and mStrawberry, tdTomato, TagRFP, TagRFP-T, and TagRFP657, and Dendra2.

In some forms, the selectable marker gene is an auxotrophic marker. Exemplary auxotrophic marker genes include but are not limited to uracil biosynthesis gene (URA3) such as orotidine-5′-phosphate decarboxylase gene, tryptophan biosynthesis gene (TRP1) such as phosphoribosylanthranilate isomerase gene, leucine biosynthesis gene (LEU2) such as β-isopropylmalate dehydrogenase gene; histidine biosynthesis gene (HIS3) such as imidazoleglycerol-phosphate dehydratase gene, and lysine biosynthesis gene (LYS2) such as α-aminoadipate reductase gene. Other suitable detectable markers include chloramphenicol acetyltransferase (CAT), luciferase lacZ (β-galactosidase), and alkaline phosphatase.

In some forms, the selectable marker gene may be separately introduced into the cell harboring the model generating system (e.g., by co-transfection, etc.). In some forms, the selectable marker gene may be linked to the CRISPR-Cas expression vector, the transposon expression vector, or both, and the marker gene expression may be controlled by a separate translation unit under an IRES (internal ribosomal entry site).

c. Termination and PolyA Sequences

One or more termination sequences and/or polyA sequences can be included in one or more components of the model generation system. In some forms, the transposon-based vector contains two stop codons operably linked to the gene of interest. In some forms, the transposase expression vector contains two stop codons operably linked to the transposase. In some forms, one stop codon of UAA or UGA is operably linked to the transposase and/or to the gene of interest.

A poly (A) sequence, also called poly (A) tail or 3′-poly (A) tail, is understood as an adenine nucleotide sequence, for example up to 400 adenine nucleotides, by example of about 20 to about 400, preferably about 50 to about 400, more preferably about 50 to about 300, even more preferably about 50 to about 250, much more preferably about 60 to approximately 250 nucleotides of adenine. The poly (A) sequence is typically located at the 3′ end of an mRNA. Typically, a poly (A) sequence can be located within an mRNA or any other nucleic acid molecule, for example a vector, for example a vector that serves as a template for the generation of an RNA, preferably an mRNA, for example by transcription of the vector. As used herein an “effective polyA sequence” refers to either a synthetic or non-synthetic sequence that contains multiple and sequential nucleotides containing an adenine base (an A polynucleotide string) and that increases expression of the gene to which it is operably linked. A polyA sequence may be operably linked to any gene in the transposon-based vector including the target gene of interest. In some forms, a polyA sequence may be operably linked to any gene in the transposase expression vector e.g., the nucleic acid encoding the transposase. A preferred polyA sequence is optimized for use in the host animal or human and for the desired product.

### B. Animal Model of Recurrent Hepatocellular Carcinoma (HCC)

As described in the non-limiting Examples, a mouse model harboring liver cell specific knockdown of TP53 and cMyc overexpression has been established. Thus, a non-human animal model of recurrent hepatocellular carcinoma (HCC) is provided. The non-human animal model is suitable for research purposes such as investigating the molecular and genetic mechanisms underlying recurrent HCC tumor development, identifying potential therapeutic targets, and testing potential compounds for the treatment of recurrent HCC.

The non-human animal model of recurrent HCC generally contains one or more genetic modifications introduced by integration of the model generating system described in Section II(A). When the model generating system is stably integrated into the target liver cells of the target animal, the model generating system modifies expression of Tp53 and expresses the cMyc oncogene in the target liver cells of the target animal, resulting in the development of a focal HCC tumor in the liver of the target animal.

Thus, the non-human animal model is a genetically modified non-human animal. As used herein, the term “genetically modified non-human animal” refers to a non-human animal having exogenous DNA in at least one chromosome of the animal's genome. In some forms, at least one or more cells, e.g., at least 1%, 2%, 3%, 4%, 5%, 10%, 20%, 30%, 40%, 50% of cells of the genetically modified non-human animal have the exogenous DNA in its genome. The cell having exogenous DNA, i.e., the target cells, can be various kinds of cells, e.g., an endogenous cell, a somatic cell, an immune cell, a T cell, a B cell, or an endogenous tumor cell. In some forms, genetically modified non-human animals contain a modified endogenous locus that contains an exogenous sequence (e.g., a human sequence), e.g., a replacement of one or more non-human sequences with one or more human sequences. The non-human animals are generally not able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some forms, the chimeric gene or chimeric nucleic acid has at least one portion of the sequence that is derived from two or more different sources, e.g., sequences encoding different proteins or sequences encoding the same (or homologous) protein of two or more different species. In some forms, the chimeric gene or the chimeric nucleic acid is a humanized gene or humanized nucleic acid.

In some forms, the target cells are liver cells. Exemplary target liver cells include but are not limited to hepatocytes, Kupffer cells, stellate (Ito) cells, sinusoidal endothelial cells (SECs), cholangiocytes, biliary epithelial cells (BECs), liver progenitor cells (LPCs), pit cells or liver-associated natural killer (NK) cells, dendritic cells, and liver sinusoidal endothelial cell (LSEC) fenestrations. In preferred forms, the target liver cells are hepatocytes.

The non-human animal model develops a focal HCC tumor after a period of time following integration of the model generating system. In some forms, the period of time for developing the focal HCC tumor is about 3 weeks to about 5 weeks, more preferably about 4 weeks following integration of the model generating system. Generally, the non-human animal model develops one or more recurrent HCC liver tumors after a period of time following surgical resection of the focal tumors. In some forms, the period of time for development of the recurrent HCC tumors is about six weeks to about 11 weeks following surgical resection of the focal tumors. In some forms, the period of time for development of the recurrent HCC tumors is about six weeks to about seven months following surgical resection of the focal tumors. In some forms, the period of time for development of the recurrent HCC tumors is about six weeks, about two months, about three months, about four months, about 5 months, about six months, about seven months following surgical resection of the focal tumors.

Recurrent HCC tumor cells and tissues are defined structurally and functionally as described herein; using methods and assays similar to those described below. Because recurrent HCC tumor cells and tissues are known to evolve phenotypically and functionally over time as additional genetic mutations occur, the recurrent HCC tumor cells and tissues may change phenotypically and functionally over time in the non-human animal model. Nevertheless, one can use the methods as described herein and employ the markers as disclosed herein, to consistently isolate and/or identify recurrent HCC tumor cells and tissues. In some forms, the HCC tumor cells and tissues express cell surface markers including but not limited to Alpha-fetoprotein and glypican 3.

Generally, the non-human animal model is an immune-competent animal. An “immune-competent” animal, as used herein, is one in which the native or natural innate and adaptive immune system has been retained (i.e., not artificially altered), such that the animal retains its normal capacity to develop an immune response against a foreign antigen. One advantage is that the animal models have been tolerized specifically to certain xenogenic cells and/or tissues e.g., cells and/or tissues produced by the model generating system, such that they do not recognize such xenogenic cells and/or tissues as foreign cells and/or tissues. Therefore, cells and/or tissues produced by the model-generating system can be achieved without the need to resort to germline genetic modification of the recipient animal's native immune system or the use of immunosuppressive agents to prevent the animal from rejecting the xenogenic cells and/or tissues. A further advantage is that the animal remains capable of mounting a normal immune response against the cells and/or tissues produced by the model generating system. Thus, such immune-competent animals would usually have T, B, and/or NK cells that are normal and/or unaltered (insofar as being not artificially altered or engineered, it being appreciated that a natural mutation may arise, which otherwise does not significantly impair the animal's normal immune response). The non-human animal model is immunologically tolerant to the HCC tumor cells, while maintaining a competent immune system. The animal models are “tolerogenic” to the HCC tumor cells, which means they are immunologically tolerant such that they maintain a state of tolerance to the HCC tumor cells, instead of mounting an immune response to, or rejection of, the HCC tumor cells. The term “tolerogenic,” as referred to herein, generally means that the animals tolerate the HCC tumors, without being immunocompromised or immunodeficient, either through the use of germline genetic modifications or immunosuppressive agents. This is in contrast with an immunodeficient or immunocompromised animal where the natural or native immune response is attenuated, weakened, or decreased, such that the animal has an altered immunocompetence to fight a foreign antigen. Such animals usually have atypical T, B, and/or NK cells. In preferred forms, the tumors are derived from the immune competent mice; therefore, the tumors are not rejected by the immune system of the mice.

The target animal for harboring the model-generating system is preferably a mammal. In some forms, the mammal is a rodent such as mice, rats, squirrels, prairie dogs, porcupines, beavers, guinea pigs, and hamsters. In some forms, the target animal is a rabbit. In some forms, the target animal is zebra fish. In preferred forms, target animal is a rodent, preferably mice or rats. The target animal chosen will various uses for modeling and studying human disease and evaluation of treatments. In one or more forms, the target animals are useful as models for recurrent HCC, and/or methods of modeling recurrent HCC using the model-generating system described above. For example, HCC tumors could be established in the recipient animal via the model generating system for testing of pharmaceuticals, cytotherapy, photodynamic therapy, magnetic hyperthermic therapy, gene therapy, and the like. The model generating system can be used to promote the development of the HCC tumor cells and/or tissue specifically in liver. For this reason, it is desirable to restrict movement by the tumor cells to facilitate tumor formation in the liver.

In some forms, the non-human animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some forms, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 12959/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some forms, the mouse is a mix of the 129 strain and the C57BL/6 strain. In some forms, the mouse is a mix of the 129 strains, or a mix of the BL/6 strains. In some forms, the mouse is a BALB strain, e.g., BALB/c strain. In some forms, the mouse is a mix of a BALB strain and another strain. In some embodiments, the mouse is from a hybrid line (e.g., 50% BALB/c-50% 12954/Sv; or 50% C57BL/6-50% 129).

In some forms, the non-human animal is a rat. The rat can be selected from a Wistar rat, an LEA strain, a Sprague Dawley strain, a Fischer strain, F344, F6, and Dark Agouti. In some forms, the rat strain is a mix of two or more strains selected from the group consisting of Wistar, LEA, Sprague Dawley, Fischer, F344, F6, and Dark Agouti.

### C. Kits

Materials and reagents for preparing the model-generating system described above can be packaged together in any suitable combination as a kit useful for performing, or aiding in the performance of, the disclosed method. It is useful if the kit components in a given kit are designed and adapted for use together in the disclosed method.

In some forms, the kit can be cell culture kit containing materials and reagents for use in the generation and/or maintenance of the non-human animals. For example, the kit can contain a CRISPR-Cas expression vector, a transposon expression vector, a transposase expression vector.

In some forms, the kit can further include reagents to aid in the transfection of the non-human animals e.g., primers for genotyping target liver cells such as primers for the Tp53 gene and primers for the c-Myc gene.

In some forms, the kits will include reagents for determining the viability and/or function of HCC tumor cells, e.g. in the presence/absence of a candidate agent, e.g. one or more antibodies that are specific for markers expressed by different types of target liver cells, or reagents for detecting particular molecules such as cytokines, etc.

Other reagents may include culture media, culture supplements, matrix compositions, and the like. In some forms, the one or more culture media may be a 1× formulation or a more concentrated formulation, for example, a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art, such as salt precipitation or selective filtration. A concentrated medium may be diluted for use with water (in certain forms, deionized and distilled) or any appropriate solution, for example, an aqueous saline solution, an aqueous buffer, or a culture medium.

The one or more media in the kit may be contained in hermetically sealed vessels which prevent contamination. Hermetically sealed vessels may be preferred for transport or storage of the culture media. The vessel may be any suitable vessel, such as a flask, a plate, a bottle, a jar, a vial, or a bag.

In addition to the above components, the kits may further include instructions for use. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc.

Yet another means would be a computer readable medium, e.g., diskette, CD, etc., on which the information has been recorded. Yet another means that may be present is a website address which may be used via the internet to access the information at a removed site.

### III. Methods of Making Non-human Animal Models of Recurrent HCC

Methods for making an animal model of recurrent hepatocellular carcinoma are also provided. In an exemplary method, the steps for making a non-human animal model of recurrent HCC include:
(i) injecting a pre-determined amount of model generating system into hepatocytes of the non-human animal;
(ii) applying an electric pulse via electroporation to the site of injection to facilitate transfection of the gene editing system into the hepatocytes, wherein the electroporation conditions are optimized to maximize genome editing efficiency;
(iii) allowing the animal to recover for a period of time during which focal HCC tumors are generated;
(iv) resecting the focal HCC tumor; and
(v) allowing the animal to recover for a period of time during which recurrent HCC tumors are established.

In some forms, the components of the model generating system are injected into the target animal in a ratio of about 10:5:1 for the CRISPR-Cas expression vector, the transposon expression vector, and the transposase expression vector, respectively.

As described above, plasmid vectors can be difficult to introduce into cells without physical treatment such as microinjection or electroporation. Therefore, in some forms, a physical delivery system is used to deliver the model generating system into the target liver cells. Physical delivery methods that can be used to facilitate transfer of the model generating system into target liver cells include but are not limited to needles, particle bombardment, microprojectile gene transfer or gene guns, electroporation, sonoporation, photoporation, magnetofection, and hydroporation, and mechanical massage.

In preferred forms, the physical delivery method is electroporation. In vivo electroporation is a gene delivery technique that is successfully used to efficiently deliver plasmid DNA to many different tissues. Tissue-specific expression of the plasmid-encoded gene or cDNA in liver cells can be obtained by administration of in vivo electroporation. The use of in vivo electroporation increases plasmid DNA uptake in liver tissues and translocate plasmids into target liver cells.

In some forms, when electroporation is used as the physical delivery method, the model generating system is delivered to the hepatocytes in two electroporation steps: a poring pulse mode and a transfer pulse mode. In some forms, the poring pulse mode of electroporation is performed at a voltage of between about 1 and about 100 volts, a pulse duration of between about 1 and about 50 milliseconds, a pulse interval of between about 1 and about 50 m/s, a pulse number of between about 1 and about 5, and a decay rate of between about 5 and about 15%. In some forms, the transfer pulse mode of electroporation is performed at a voltage of between about 1 and about 100 volts, a pulse duration of between about 1 and about 50 milliseconds, a pulse interval of between about 1 and about 50 m/s, a pulse number of between about 3 and about 8, and a decay rate of between about 30 and about 50%.

In some forms, the method of making the non-human animal model of recurrent HCC further includes the step of monitoring the target animal for the development of hepatocellular carcinoma. In these forms, monitoring includes the use of various molecular techniques to confirm the formation of HCC tumors. Suitable techniques include but are not limited to histopathology, imaging, molecular markers, and combinations thereof. In preferred forms, tumor development is monitored via palpation i.e., feeling with the fingers or hands during a physical examination. The technique or techniques employed enables the identification of one or more pathological features of recurrent HCC including but not limited to altered liver function, abnormal liver histology, and elevated liver enzymes. In preferred forms, the pathological features of recurrent HCC include elevated serum levels of one or more enzymes including but not limited to alkaline phosphatase, aspartate transferase, and albumin.

### IV. Methods of Use

The disclosed non-human animal model is valuable for research purposes such as investigating the molecular and genetic mechanisms underlying recurrent HCC tumor development, identifying potential therapeutic targets, and evaluating/screening potential compounds for the treatment of recurrent HCC. The disclosed non-human animal model is also valuable for the collection of liver cells and/or tissues containing recurrent HCC tumors for research purposes.

### A. Providing Cells/Tissues Containing Recurrent HCC Tumors

In some forms, the non-human animal model and methods can be used to generate liver cells with mutations in target genes for disease modeling studies, small molecule production, and/or drug discovery.

In some forms, the liver cells harboring recurrent HCC tumors may have one or more molecular markers similar to focal HCC tumors. Exemplary molecular markers of recurrent HCC tumors include but are not limited to serum albumin and serum glypican 3.

### B. Drug Screening and Evaluation

There is a lack of effective treatments available for the treatment of recurrent HCC. The disclosed compositions and methods are useful for investigating the activity or efficacy of one or more test compounds to treat recurrent HCC.

An exemplary method of screening and evaluating compounds for treating recurrent hepatocellular carcinoma (HCC) one or more of the following steps:
(i) administering a pre-determined concentration of a test compound to the non-human animal or to HCC tumor cells harvested from the non-human animal;
(ii) incubating the animal or cells for a pre-determined period of time;
(iii) measuring one or more parameters indicative of cellular response to the test compound; and
(iv) assessing the efficacy of the test compound by comparing the measured parameters of step (iii) to measured parameters of an untreated control non-human animal, wherein a significant difference between the measured parameters of step (iii) and the measured parameters of the untreated control indicates that the test compound can be considered for treating recurrent HCC.

In some forms, the one or more parameters indicative of cellular response are selected from the group consisting of cell viability, cell proliferation, cell death, metabolic activity, gene expression, protein expression, and any combination thereof.

In some forms, the measuring of the one or more parameters in step (iii) is performed using one or more techniques such as flow cytometry, microscopy, gene expression analysis, quantitative polymerase chain reaction (qPCR), enzyme-linked immunosorbent assay (ELISA), mass spectrometry, and any combination thereof.

Methods for screening test compounds are known in the art and further include but are not limited to scintillation proximity assays, Direct fluorescence measurement, Fluorescence polarization, Fluorescence resonance energy transfer (FRET), Time-resolved fluorescence (TRF, HTRF, and TiRF), AlphaScreen, Highcontent screening (HCS), Protein fragment complementation assays (PCA), microfluidics, and label-free technologies (reviewed in Janzen (2014) Chemistry and Biology, 21(9), pages 1162-1170).

In some forms, the predetermined concentration and predetermined period of time in steps (i) and (ii), respectively, are optimized based on the type of test compound. In some forms, the test compound is selected from a library of compounds containing natural products, synthetic compounds, or a combination thereof.

In some forms, the test compound could be an existing FDA-approved drug, with known indications. In some forms, the test compound is an unknown compound that has not been reported for its anti-tumoral effect. In some forms, the test compound could be one or more chemically synthesized compounds. In some forms, the test compound could be one or more components extracted from natural plants or herbs. In some forms, the test compound is a drug used to treat liver cancer e.g., Sorafenib, Lenvatinib, Regorafenib, Cabozantinib, Pembrolizumab, Atezolizumab, Bevacizumab, Nivolumab, Ipilimumab.

In some forms, the non-human animal model of recurrent HCC and cells/tissues thereof can also be used to evaluate both general and targeted therapies. For example, a general therapy can be a pharmaceutical or chemical with physiological effects, such as pharmaceuticals that have been used in chemotherapy for cancer. Chemotherapeutic agents inhibit proliferation of tumor cells, and generally interfere with DNA replication or cellular metabolism. In some forms, the non-human animal model can be used to characterize the effects of chemotherapeutic agents on tumor-bearing cells, healthy cells, and the animal harboring the HCC tumor. In some forms, the therapy is a targeted therapy. A targeted therapy refers to a therapy that directly interferes with a specific gene. Preferably, a targeted therapy directly interferes with the expression of a gene involved in recurrent HCC. The effectiveness of a targeted therapy can be determined by the ability of the therapy to inhibit an oncogene or activate a tumor suppressor gene.

### C. Biomarker Identification

Understanding of the molecular mechanisms driving HCC recurrence is scarce due to a combination of factors including lack of a cell line that truly distinguishes recurrent HCC from primary HCC, and lack of a reliable translational mouse model with an intact immune system that could recapitulate the process of HCC recurrence. The disclosed non-human animal model and/or cells/tissues therefrom are ideal for investigation and identification of biomarkers that predict tumor recurrence, and identification of molecular targets for the treatment of recurrent HCC.

Methods of using animal models for the identification of biomarkers and potential molecular targets are known in the art. An exemplary method for identifying biomarkers of hepatocellular carcinoma (HCC), includes one or more of the following steps:
(i) extracting recurrent HCC tumor tissue from the non-human animal model of recurrent HCC;
(ii) isolating and purifying nucleic acids, proteins, and/or metabolites from the recurrent HCC tumor tissue;
(iii) measuring the expression level of the nucleic acids, proteins, and/or metabolites;
(iv) comparing the expression levels of the nucleic acids, proteins, and/or metabolites to corresponding expression levels in the nucleic acids, proteins, and/or metabolites of control liver issue; and
(v) identifying one or more biomarkers of HCC based on statistically significant differences in expression levels of the nucleic acids, proteins, and/or metabolites between the recurrent HCC tumor tissue and the control liver issue.

Methods of extracting and analyzing nucleic acids, proteins, or metabolites from tumor tissue are known in the art. Suitable methods include but are not limited to PCR, qPCR, RNA-seq, mass spectrometry, ELISA, Western blot, immunohistochemistry, and metabolic profiling., and any combination thereof. Unless otherwise specified, the practice of the methods described herein can take advantage of the techniques of cell biology, cell culture, molecular biology, transgenic biology, microbiology, recombinant DNA, and immunology. These techniques are explained in detail in the following literature, for example: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered, 1985); Oligonucleotide Synthesis (M. J. Gaited., 1984); Mullis et al U.S. Pat. No. 4,683,195; Nucleic Acid Hybridization (B. D. Hames& S. J. Higginseds. 1984); Transcription and Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In Enzymology (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986), each of which is incorporated herein in its entirety by reference.

In some forms, the methods of biomarker identification include profiling the genome of HCC tumor cells, wherein the tumor cells are from the non-human animal model of recurrent HCC. An exemplary genome analysis includes performing segmentation analysis of the genome profile, identifying loci, and prioritizing the identified loci; and relating to recurrent HCC querying genes at the identified loci. In some forms, gene queries at identified loci are based on gene expression data. In some forms, gene queries at the identified locus are based on an in vitro screening assay.

The identified biomarker is generally a biomarker associated with recurrent HCC. The identified biomarker can be a diagnostic biomarker, a prognostic biomarker, or a pharmacogenomic biomarker. In some forms, identified biomarker is a nucleic acid biomarker or a protein biomarker.

The present invention can be further understood by the following non-limiting paragraphs:

1. A recurrent hepatocellular carcinoma (HCC) model generating system containing the following components:
(i) a CRISPR-Cas expression vector containing:
a nucleic acid sequence encoding a CRISPR-associated (Cas) endonuclease;
a nucleic acid sequence encoding a single guide RNA (sgRNA), wherein the sgRNA is designed to specifically target and bind to a predetermined sequence within the Tp53 gene of a target non-human animal; and
a delivery vector carrier that facilitates the delivery and expression of the sequences encoding the Cas endonuclease and the sgRNA in target liver cells,
wherein in the target liver cells of the target animal the combination of the Cas endonuclease and the sgRNA leads to inhibition of Tp53 expression in the target liver cells;
(ii) a transposase expression vector containing:
a nucleic acid sequence encoding a transposase; and
a first antibiotic resistance gene for the selection of cells containing the transposase expression vector,
wherein the transposase expression vector is designed to lead to expression of the transposase in the target liver cells; and
(iii) a transposon expression vector containing:
a nucleic acid sequence encoding an oncogene;
a pair of inverted terminal repeat (ITR) sequences located on either side of the sequence encoding the oncogene, wherein the ITR sequences are recognized and bound by the transposase; and
a second antibiotic resistance gene for the selection of cells containing the transposon expression vector,
wherein the transposon expression vector is designed to lead to expression of the oncogene in the target liver cells.

2. The model generating system of paragraph 1, wherein the oncogene is selected from the group containing cellular myelocytomatosis (c-myc), telomerase reverse transcriptase (TERT), Rat sarcoma (RAS), and CTNNB1.

3. The model generating system of paragraph 2, wherein the oncogene contains SEQ ID NO:2 or a variant thereof.

4. The model generating system of any one of paragraphs 1-3, wherein the sgRNA molecule contains SEQ ID NO:1 or a variant thereof.

5. The model generating system of any one of paragraphs 1-4, wherein the Cas endonuclease is selected from the group consisting of Cas9, Cas12a, Cas12b, and Cas13.

6. The model generating system of any one of paragraphs 1-5, wherein the CRISPR-Cas expression vector further contains one or more transcriptional mediation sequences selected from the group containing nuclear localization sequences, promoter sequences, enhancer sequences, marker sequences, termination signal sequences, polyadenylation signal sequences, and splicing signal sequences.

7. The model generating system of any one of paragraphs 1-6, wherein the delivery vector carrier is a plasmid, viral vector, or non-viral vector.

8. The model generating system of any one of paragraphs 1-7, wherein the CRISPR-Cas expression vector further contains one or more selectable markers for identifying and isolating transfected or transduced cells.

9. The model generating system of any one of paragraphs 1-8, wherein the transposase is selected from the group consisting of Sleeping Beauty, piggyBac, Tn5, Tn7, and Mu.

10. The model generating system of any one of paragraphs 1-9, wherein the inverted terminal repeat sequences are selected from the group consisting of Sleeping Beauty, piggyBac, Tn5, Tn7, and Mu ITR sequences.

11. The model generating system of any one of paragraphs 1-10, wherein the first and second antibiotic resistance genes are independently selected from the group consisting of ampicillin, kanamycin, tetracycline, and chloramphenicol resistance genes.

12. The model generating system of any one of paragraphs 1-11, wherein the transposon expression vector further contains a regulatory element for controlling the expression of the oncogene in a host cell, wherein the regulatory element is selected from the group consisting of promoters, enhancers, silencers, and insulators.

13. The model generating system of paragraph 12, wherein the promoter is a liver-specific promoter selected from the group consisting of albumin promoter, the alpha-1 antitrypsin promoter, the apolipoprotein E promoter, alpha-fetoprotein (AFP) promoter, CYP1A1 promoter, CYP2B6 promoter, CYP3A4 promoter, fatty acid-binding protein (FABP) promoter, glucose-6-phosphatase (G6Pase) promoter, Hepatitis B virus (HBV) core promoter, HNF1-alpha promoter, HNF4-alpha promoter, Interleukin-6 (IL-6) promoter, and Transferrin promoter.

14. The model generating system of any one of paragraphs 1-13, wherein the transposon expression vector further contains a reporter gene sequence located within the ITR sequence for monitoring the expression of the oncogene in a host cell.

15. The model generating system of paragraph 14, wherein the reporter gene sequence encodes a protein selected from the group consisting of green fluorescent protein (GFP), red fluorescent protein (RFP), luciferase, and β-galactosidase.

16. The model generating system of any one of paragraphs 1-15, wherein delivery of the transposon expression vector to the host cell increases c-myc expression in the host cell.

17. A non-human animal model of recurrent hepatocellular carcinoma (HCC) containing a genetic modification introduced by integration of the model generating system of any of paragraphs 1-16 into the target liver cells of the target animal, wherein, when the model generating system is stably integrated into the target liver cells of the target animal, the model generating system modifies expression of Tp53 and expresses the oncogene in the target liver cells of the target animal, resulting in the development of a focal HCC tumor in the liver of the target animal.

18. The non-human animal model of paragraph 17, wherein the period of time for the development of a focal HCC tumor is about 4 weeks.

19. The non-human animal model of paragraph 17 or 18, wherein the non-human animal model develops one or more recurrent HCC liver tumors after a period of time following surgical resection of the focal tumors.

20. The non-human animal model of paragraph 19, wherein the period of time for development of the recurrent HCC tumors is about 6 weeks to about 6 months following surgical resection of the focal tumors.

21. The non-human animal model of any one of paragraphs 17-20, wherein the target animal is a mammal, preferably mice, rats, or other rodents.

22. The non-human animal model of paragraph 21, wherein the mice are immune-competent mice.

23. A method of generating a non-human animal model of recurrent hepatocellular carcinoma (HCC), containing the steps of:
(i) injecting pre-determined amounts of the components of the model generating system of paragraphs 1-17 into hepatocytes of the target animal;
(ii) applying an electric pulse via electroporation to the site of injection to facilitate transfection of the model generating system into the hepatocytes, wherein conditions of the electroporation are optimized to maximize transfection efficiency;
(iii) allowing the target animal to recover for a period of time during which a focal HCC tumor is generated;
(iv) resecting the focal HCC tumor; and
(v) allowing the target animal to recover for a period of time during which recurrent HCC tumors are established.

24. The method of paragraph 23, wherein the components of the model generating system injected into the target animal in a ratio from about 20:5:1 to about 0:5:1, preferably of about 10:5:1, for the CRISPR-Cas expression vector, the transposon expression vector, and the transposase expression vector, respectively.

25. The method of paragraph 23 or 24, wherein the model generating system is delivered to the hepatocytes in two electroporation steps containing a poring pulse mode and a transfer pulse mode.

26. The method of paragraph 25, wherein the poring pulse mode of electroporation is performed at a voltage of between about 1 and about 100 volts, a pulse duration of between about 1 and about 50 milliseconds, a pulse interval of between about 1 and about 50 m/s, a pulse number of between about 1 and about 5, and a decay rate of between about 5 and about 15%.

27. The method of paragraph 25 or 26, wherein the transfer pulse mode of electroporation is performed at a voltage of between about 1 and about 100 volts, a pulse duration of between about 1 and about 50 milliseconds, a pulse interval of between about 1 and about 50 m/s, a pulse number of between about 3 and about 8, and a decay rate of between about 30 and about 50%.

28. The method of any one of paragraphs 23-27 further containing the step of monitoring the target animal for the development of hepatocellular carcinoma, wherein the monitoring includes the use of a technique selected from the group consisting of histopathology, imaging, molecular markers, and combinations thereof.

29. The method of any one of paragraphs 23-28, wherein the non-human animal model of recurrent HCC exhibits one or more pathological features of recurrent HCC selected from the group consisting of altered liver function, abnormal liver histology, and elevated liver enzymes selected from the group containing alkaline phosphatase, aspartate transferase, albumin.

30. A method of screening and evaluating compounds for treating recurrent hepatocellular carcinoma (HCC), containing the steps of:
(i) administering a pre-determined concentration of a test compound to the non-human animal or to HCC tumor cells harvested from the non-human animal of any one of paragraphs 17-29;
(ii) incubating the animal or cells for a pre-determined period of time; (iii) measuring one or more parameters indicative of cellular response to the test compound; and
(iv) assessing the efficacy of the test compound by comparing the measured parameters of step (iii) to measured parameters of an untreated control non-human animal, wherein a significant difference between the measured parameters of step (iii) and the measured parameters of the untreated control indicates that the test compound can be considered for treating recurrent HCC.

31. The method of paragraph 30, wherein the one or more parameters indicative of cellular response are selected from the group consisting of cell viability, cell proliferation, cell death, metabolic activity, gene expression, protein expression, and any combination thereof.

32. The method of paragraph 30 or 31, wherein the measuring of the one or more parameters in step (iii) is performed using one or more techniques selected from the group consisting of flow cytometry, microscopy, gene expression analysis, quantitative polymerase chain reaction (qPCR), enzyme-linked immunosorbent assay (ELISA), mass spectrometry, and any combination thereof.

33. The method of any one of paragraphs 30-32, wherein the predetermined concentration and predetermined period of time in steps (i) and (ii), respectively, are optimized based on the type of test compound.

34. The method of any one of paragraphs 30-33, wherein the test compound is selected from a library of compounds containing natural products, synthetic compounds, or a combination thereof.

35. A method for identifying biomarkers of hepatocellular carcinoma (HCC), the method containing the steps of:
(i) extracting recurrent HCC tumor tissue from the non-human animal model of any of paragraphs 17-29;
(ii) isolating and purifying nucleic acids, proteins, and/or metabolites from the recurrent HCC tumor tissue;
(iii) measuring the expression level of the nucleic acids, proteins, and/or metabolites;
(iv) comparing the expression levels of the nucleic acids, proteins, and/or metabolites to corresponding expression levels in the nucleic acids, proteins, and/or metabolites of control liver issue; and
(v) identifying one or more biomarkers of HCC based on statistically significant differences in expression levels of the nucleic acids, proteins, and/or metabolites between the recurrent HCC tumor tissue and the control liver issue.

36. The method of paragraph 35, wherein extracting and analyzing nucleic acids, proteins, or metabolites from the recurrent HCC tumor tissue contains utilizing techniques selected from the group consisting of PCR, qPCR, RNA-seq, mass spectrometry, ELISA, Western blot, immunohistochemistry, and metabolic profiling.

37. A recurrent hepatocellular carcinoma (HCC) model generating system containing the following components:
(i) a CRISPR-Cas expression vector containing:
a nucleic acid sequence encoding a CRISPR-associated (Cas) endonuclease;
a nucleic acid sequence encoding a single guide RNA (sgRNA), wherein the sgRNA is designed to specifically target and bind to a predetermined sequence within a first oncogene of a target non-human animal; and
a delivery vector carrier that facilitates the delivery and expression of the sequences encoding the Cas endonuclease and the sgRNA in target liver cells,
wherein in the target liver cells of the target animal the combination of the Cas endonuclease and the sgRNA leads to inhibition of expression of the first oncogene in the target liver cells;
(ii) a transposase expression vector containing:
a nucleic acid sequence encoding a transposase; and
a first antibiotic resistance gene for the selection of cells containing the transposase expression vector,
wherein the transposase expression vector is designed to lead to expression of the transposase in the target liver cells; and
(iii) a transposon expression vector containing:
a nucleic acid sequence encoding a second oncogene;
a pair of inverted terminal repeat (ITR) sequences located on either side of the sequence encoding the second oncogene, wherein the ITR sequences are recognized and bound by the transposase; and
a second antibiotic resistance gene for the selection of cells containing the transposon expression vector,
wherein the transposon expression vector is designed to lead to expression of the second oncogene in the target liver cells.

38. The model generating system of paragraph 37, wherein the first oncogene is selected from the group containing Tumor protein p53 (TP53), TSC Complex subunit 2 (Tsc2), Kelch-like ECH-associated protein 1 (KEAP1), Adenomatous polyposis coli (Apc), Phosphatase and tensin homolog (PTEN), telomerase reverse transcriptase (TERT), Rat sarcoma (RAS), Catenin Beta 1 (CTNNB1); and
wherein the second oncogene is selected from the group containing cellular myelocytomatosis (c-myc) and CTNNB1.

39. The model generating system of paragraph 37 or 38, wherein the first oncogene is Tumor protein p53 (TP53), and the second oncogene is c-myc.

40. The model generating system of paragraph 37 or 38, wherein the first oncogene is Tsc2 and the second oncogene is c-myc.

41. The model generating system of paragraph 37 or 38, wherein the first oncogene is Pten and the second oncogene is c-myc.

42. The model generating system of paragraph 37 or 38, wherein the first oncogene is Keap1 and the second oncogene is c-myc.

43. The model generating system of paragraph 37 or 38, wherein the first oncogene is CTTNB1 and the second oncogene is c-myc.

### Examples

### Materials and Methods

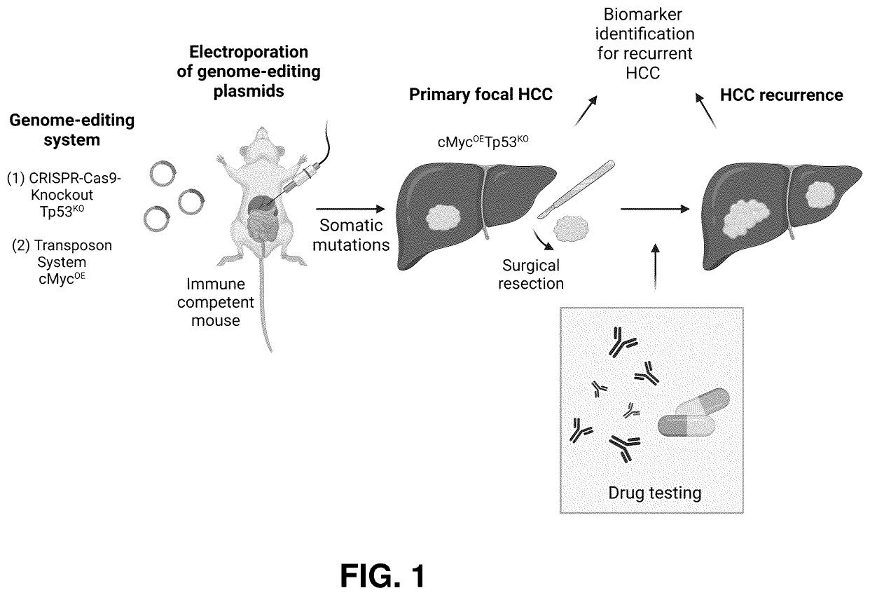

FIG. 1 is a schematic showing the experimental procedure. Briefly, a genome-editing DNA plasmid mix is injected directly into a pre-determined location of the left lobes of the liver of immune competent mice followed by electroporation. The genome editing plasmids contain CRISPR-Cas9 KO and Sleeping Beauty transposon systems, which knock out (KO) Tp53 (tumor suppressor) and over-express cMyc (oncogene) respectfully, in specific lobes of livers of immune-competent mice. The electric pulse mediates the transfection of hepatocytes, causing somatic genome-editing and cancer cell transformation. Mice were sutured and allowed to recover. HCC tumors develop in as early as 4 weeks depending on the genetic alterations. Focal tumors were established. Surgical resection (hepatectomy) was performed to remove primary HCC.

### Induction of Hepatocellular Carcinoma (HCC)

A midline incision was made at the abdomen of the C57BL/6 male mouse (8-10 weeks old) to expose the liver. 32 μg in 25 μL of genome editing plasmids was injected into the left lateral lobe of the liver from mouse with a syringe. The genome-editing plasmids contained three (3) major components (10:5:1 ratio): (i) a CRISPR-Cas9 plasmid with Cas9 gene and single-guided RNAs (sgRNAs) targeting the Tp53 sequence (sgTp53 sequence: CCTCGAGCTCCCTCTGAGCC (SEQ ID NO:1)); (ii) a transposon plasmid with promoter driving oncogene c-MYC (Myc sequence):

(SEQ ID NO: 2)
ATGCCCCTCAACGTTAGCTTCACCAACAGGAACTATGACCTCGACTACG
ACTCGGTGCAGCCGTATTTCTACTGCGACGAGGAGGAGAACTTCTACCA
GCAGCAGCAGCAGAGCGAGCTGCAGCCCCCGGCGCCCAGCGAGGATATC
TGGAAGAAATTCGAGCTGCTGCCCACCCCGCCCCTGTCCCCTAGCCGCC
GCTCCGGGCTCTGCTCGCCCTCCTACGTTGCGGTCACACCCTTCTCCCT
TCGGGGAGACAACGACGGCGGTGGCGGGAGCTTCTCCACGGCCGACCAG
CTGGAGATGGTGACCGAGCTGCTGGGAGGAGACATGGTGAACCAGAGTT
TCATCTGCGACCCGGACGACGAGACCTTCATCAAAAACATCATCATCCA
GGACTGTATGTGGAGCGGCTTCTCGGCCGCCGCCAAGCTCGTCTCAGAG
AAGCTGGCCTCCTACCAGGCTGCGCGCAAAGACAGCGGCAGCCCGAACC
CCGCCCGCGGCCACAGCGTCTGCTCCACCTCCAGCTTGTACCTGCAGGA
TCTGAGCGCCGCCGCCTCAGAGTGCATCGACCCCTCGGTGGTCTTCCCC
TACCCTCTCAACGACAGCAGCTCGCCCAAGTCCTGCGCCTCGCAAGACT
CCAGCGCCTTCTCTCCGTCCTCGGATTCTCTGCTCTCCTCGACGGAGTC
CTCCCCGCAGGGCAGCCCCGAGCCCCTGGTGCTCCATGAGGAGACACCG
CCCACCACCAGCAGCGACTCTGAGGAGGAACAAGAAGATGAGGAAGAAA
TCGATGTTGTTTCTGTGGAAAAGAGGCAGGCTCCTGGCAAAAGGTCAGA
GTCTGGATCACCTTCTGCTGGAGGCCACAGCAAACCTCCTCACAGCCCA
CTGGTCCTCAAGAGGTGCCACGTCTCCACACATCAGCACAACTACGCAG
CGCCTCCCTCCACTCGGAAGGACTATCCTGCTGCCAAGAGGGTCAAGTT
GGACAGTGTCAGAGTCCTGAGACAGATCAGCAACAACCGAAAATGCACC
AGCCCCAGGTCCTCGGACACCGAGGAGAATGTCAAGAGGCGAACACACA
ACGTCTTGGAGCGCCAGAGGAGGAACGAGCTAAAACGGAGCTTTTTTGC
CCTGCGTGACCAGATCCCGGAGTTGGAAAACAATGAAAAGGCCCCCAAG
GTAGTTATCCTTAAAAAAGCCACAGCATACATCCTGTCCGTCCAAGCAG
AGGAGCAAAAGCTCATTTCTGAAGAGGACTTGTTGCGGAAACGACGAGA
ACAGTTGAAACACAAACTTGAACAGCTACGGAACTCTTGTGCGGACCCA
GCTTTCTTGTACAAAGTGGTTGATATCCAGCACAGTGGCGGCCGCTCGA
GTCTAGAGGGCCCGCGGTTCGAAGGTAAGCCTATCCCTAACCCTCTCCT
CGGTCTCGATTCTACGCGTACCGGTTAG);

and (iii) a transposase enzyme.

An electric pulse was delivered to the injection site. The parameters for the electric pulse were as follows. In poring pulse mode, 3 electric pulses were delivered with pulse voltage of 50V, pulse length of 30 m/s, pulse interval of 50 m/s, decay rate of 10%, +polarity. In transfer pulse mode, 5 electric pulses were delivered with pulse voltage of 15V, pulse length of 50 m/s, pulse interval of 50 m/s, decay rate of 40%, +/−polarity. The mouse was sutured and monitored for recovery.

### Hepatectomy or Resection of Primary HCC

The mice were housed and monitored for 6-8 weeks. After primary HCC was formed, a midline incision was made to expose the liver with primary HCC. Tumor development is monitored via palpation i.e., feeling with the fingers or hands during a physical examination. A silk thread was used to make a knot on the base of the left lateral lobe, close to the liver hilum. The tied lobe was removed using microsurgery scissors right above the knot. The mice were monitored closely after suture for recovery.

### Results

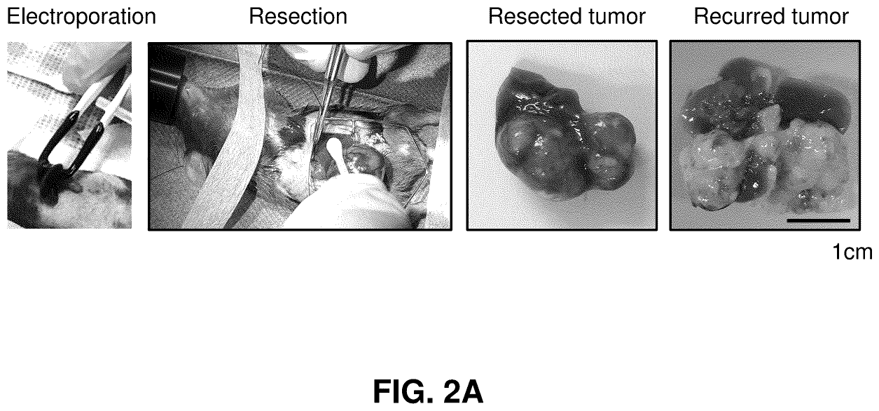

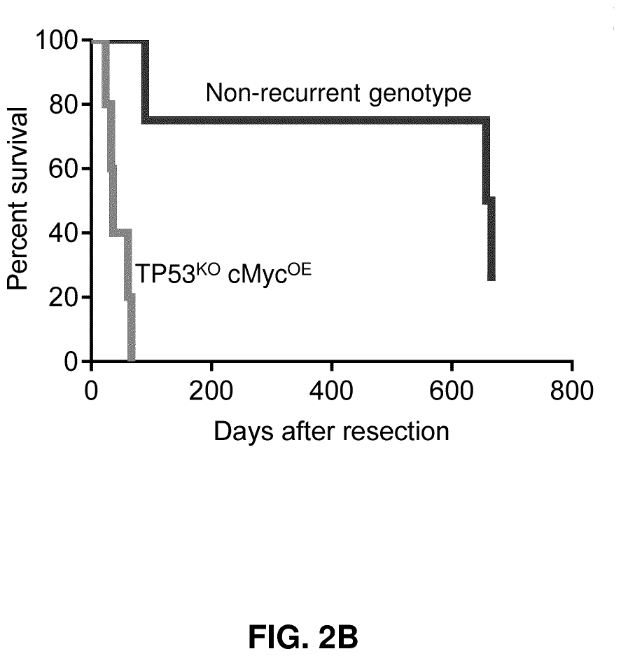

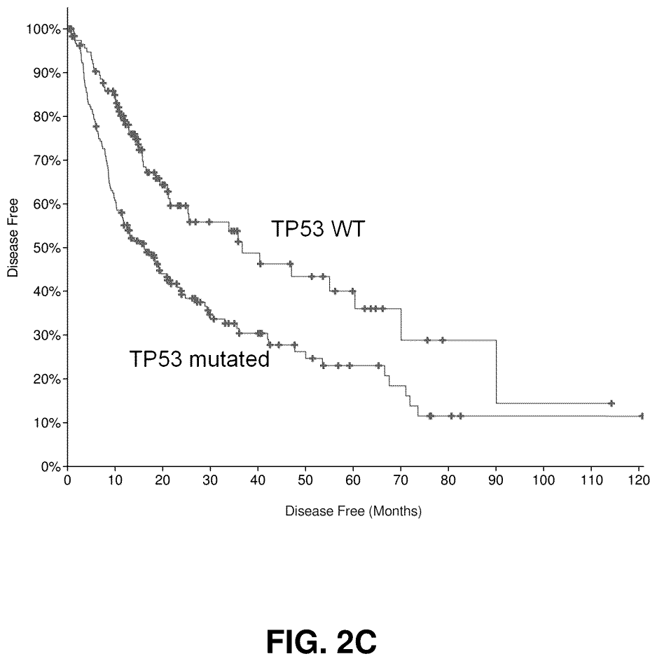

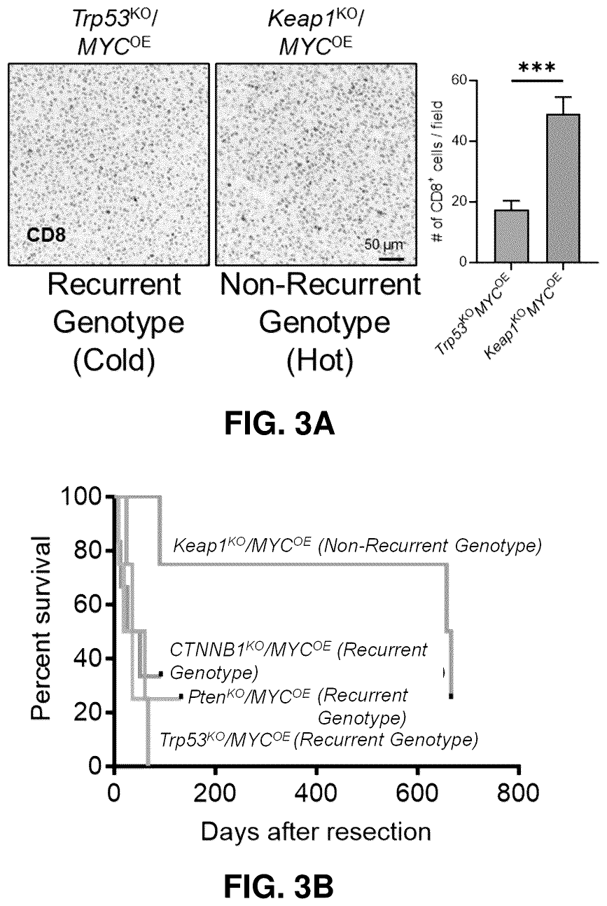

All mice survived following surgical removal of HCC. Recurrent HCC tumors formed after 6-8 weeks of surgical resection (FIG. 2A). TCGA data revealed that HCC patients with TP53 mutations have poorer disease-free survival post-resection as compared to HCC patients with TP53 WT (FIG. 2C).

### CONCLUSION

HCC tumors (Tp53^{KO}cMyc^{OE}) which are highly recurrent post-surgical resection were generated in the current study. These results suggest that the current model is a reliable recurrent mouse HCC model. This mouse model can be used to test new drugs and identify new biomarkers.

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Those skilled in the art will recognize, or be able to ascertain, using no more than routine experimentation, many equivalents to the specific embodiments of the method and compositions described herein. Such equivalents are intended to be encompassed by the following claims.

We claim:

1. A recurrent hepatocellular carcinoma (HCC) model generating system comprising the following components:
(i) a CRISPR-Cas expression vector comprising:
a nucleic acid sequence encoding a CRISPR-associated (Cas) endonuclease;
a nucleic acid sequence encoding a single guide RNA (sgRNA), wherein the sgRNA is designed to specifically target and bind to a predetermined sequence within a Tp53 gene of a target non-human animal; and
a delivery vector carrier that facilitates the delivery and expression of the sequences encoding the Cas endonuclease and the sgRNA in target liver cells,
wherein in the target liver cells of the target animal the combination of the Cas endonuclease and the sgRNA leads to inhibition of expression of the Tp53 gene in the target liver cells;
(ii) a transposase expression vector comprising:
a nucleic acid sequence encoding a transposase; and
a first antibiotic resistance gene for the selection of cells containing the transposase expression vector,
wherein the transposase expression vector is designed to lead to expression of the transposase in the target liver cells; and
(iii) a transposon expression vector comprising:
a nucleic acid sequence encoding an oncogene;
a pair of inverted terminal repeat (ITR) sequences located on either side of the sequence encoding the oncogene, wherein the ITR sequences are recognized and bound by the transposase; and
a second antibiotic resistance gene for the selection of cells containing the transposon expression vector,
wherein the transposon expression vector is designed to lead to expression of the oncogene in the target liver cells.

2. The model generating system of claim 1, wherein the oncogene is selected from the group comprising TSC Complex subunit 2 (Tsc2), Kelch-like ECH-associated protein 1 (KEAP1), Adenomatous polyposis coli (Apc), cellular myelocytomatosis (c-myc), Phosphatase and tensin homolog (PTEN), telomerase reverse transcriptase (TERT), Rat sarcoma (RAS), and Catenin Beta 1 (CTNNB1).

3. The model generating system of claim 1, wherein the sgRNA molecule comprises SEQ ID NO:1 or a variant thereof.

4. The model generating system of claim 1, wherein the Cas endonuclease is selected from the group consisting of Cas9, Cas12a, Cas12b, and Cas13.

5. The model generating system of claim 1, wherein the CRISPR-Cas expression vector further comprises one or more transcriptional mediation sequences selected from the group comprising nuclear localization sequences, promoter sequences, enhancer sequences, marker sequences, termination signal sequences, polyadenylation signal sequences, splicing signal sequences, or selectable markers for identifying and isolating transfected or transduced cells.

6. The model generating system of claim 1, wherein the delivery vector carrier is a plasmid, viral vector, or non-viral vector.

7. The model generating system of claim 1, wherein the transposase is selected from the group consisting of Sleeping Beauty, piggyBac, Tn5, Tn7, and Mu.

8. The model generating system of claim 1, wherein the inverted terminal repeat sequences are selected from the group consisting of Sleeping Beauty, piggyBac, Tn5, Tn7, and Mu ITR sequences.

9. The model generating system of claim 1, wherein the first and second antibiotic resistance genes are independently selected from the group consisting of ampicillin, kanamycin, tetracycline, and chloramphenicol resistance genes.

10. The model generating system of claim 1, wherein the transposon expression vector further comprises a regulatory element for controlling the expression of the second oncogene in a host cell, wherein the regulatory element is selected from the group consisting of promoters, enhancers, silencers, and insulators;
wherein the promoter is a liver-specific promoter selected from the group consisting of albumin promoter, the alpha-1 antitrypsin promoter, the apolipoprotein E promoter, alpha-fetoprotein (AFP) promoter, CYP1A1 promoter, CYP2B6 promoter, CYP3A4 promoter, fatty acid-binding protein (FABP) promoter, glucose-6-phosphatase (G6Pase) promoter, Hepatitis B virus (HBV) core promoter, HNF1-alpha promoter, HNF4-alpha promoter, Interleukin-6 (IL-6) promoter, and Transferrin promoter.

11. The model generating system of claim 1, wherein the transposon expression vector further comprises a reporter gene sequence located within the ITR sequence for monitoring the expression of the second oncogene in a host cell, wherein the reporter gene sequence encodes a protein selected from the group consisting of green fluorescent protein (GFP), red fluorescent protein (RFP), luciferase, and β-galactosidase.

12. The model generating system of claim 1, wherein delivery of the transposon expression vector to the host cell increases c-myc expression in the host cell.

13. A non-human animal model of recurrent hepatocellular carcinoma (HCC) comprising a genetic modification introduced by integration of the model generating system of claim 1 into the target liver cells of the target animal,
wherein, when the model generating system is stably integrated into the target liver cells of the target animal, the model generating system modifies expression of Tp53 and expresses the oncogene in the target liver cells of the target animal, resulting in the development of a focal HCC tumor in the liver of the target animal.

14. The non-human animal model of claim 13, wherein the period of time for the development of a focal HCC tumor is about 4 weeks;
wherein the non-human animal model develops one or more recurrent HCC liver tumors after a period of time following surgical resection of the focal tumors, and
wherein the period of time for development of the recurrent HCC tumors is about 6 weeks to about 6 months following surgical resection of the focal tumors.

15. The non-human animal model of claim 1, wherein the target animal is a mammal, preferably mice, rats, or other rodents,
wherein the mammal is an immune-competent mammal.

16. A method of generating a non-human animal model of recurrent hepatocellular carcinoma (HCC), comprising the steps of:
(i) injecting pre-determined amounts of the components of the model generating system of claim 1 into hepatocytes of the target animal;
(ii) applying an electric pulse via electroporation to the site of injection to facilitate transfection of the model generating system into the hepatocytes, wherein conditions of the electroporation are optimized to maximize transfection efficiency;
(iii) allowing the target animal to recover for a period of time during which a focal HCC tumor is generated;
(iv) resecting the focal HCC tumor; and
(v) allowing the target animal to recover for a period of time during which recurrent HCC tumors are established.

17. The method of claim 16, wherein the components of the model generating system injected into the target animal in a ratio from about 20:5:1 to about 0:5:1, preferably of about 10:5:1, for the CRISPR-Cas expression vector, the transposon expression vector, and the transposase expression vector, respectively.

18. The method of claim 16, wherein the model generating system is delivered to the hepatocytes in two electroporation steps comprising a poring pulse mode and a transfer pulse mode;
wherein the poring pulse mode of electroporation is performed at a voltage of between about 1 and about 100 volts, a pulse duration of between about 1 and about 50 milliseconds, a pulse interval of between about 1 and about 50 m/s, a pulse number of between about 1 and about 5, and a decay rate of between about 5 and about 15%; and
wherein the transfer pulse mode of electroporation is performed at a voltage of between about 1 and about 100 volts, a pulse duration of between about 1 and about 50 milliseconds, a pulse interval of between about 1 and about 50 m/s, a pulse number of between about 3 and about 8, and a decay rate of between about 30 and about 50%.

19. A method of screening and evaluating compounds for treating recurrent hepatocellular carcinoma (HCC), comprising the steps of:
(i) administering a pre-determined concentration of a test compound to the non-human animal or to HCC tumor cells harvested from the non-human animal of claim 13;
(ii) incubating the animal or cells for a pre-determined period of time;
(iii) measuring one or more parameters indicative of cellular response to the test compound; and
(iv) assessing the efficacy of the test compound by comparing the measured parameters of step (iii) to measured parameters of an untreated control non-human animal, wherein a significant difference between the measured parameters of step (iii) and the measured parameters of the untreated control indicates that the test compound can be considered for treating recurrent HCC.

20. A method for identifying biomarkers of hepatocellular carcinoma (HCC), the method comprising the steps of:
(i) extracting recurrent HCC tumor tissue from the non-human animal model of claim 13;
(ii) isolating and purifying nucleic acids, proteins, and/or metabolites from the recurrent HCC tumor tissue;
(iii) measuring the expression level of the nucleic acids, proteins, and/or metabolites;
(iv) comparing the expression levels of the nucleic acids, proteins, and/or metabolites to corresponding expression levels in the nucleic acids, proteins, and/or metabolites of control liver issue; and
(v) identifying one or more biomarkers of HCC based on statistically significant differences in expression levels of the nucleic acids, proteins, and/or metabolites between the recurrent HCC tumor tissue and the control liver issue.
